(19) United States
(12) Patent Application Publication
(10) Pub. No.: US 2003/0003482 A1
(43) Pub. Date: Jan. 2, 2003

(54) MRP8/MRP14 heterodimer, or its individual components in combination, for treating and/or preventing skin diseases, wounds and/or wound-healing disturbances, having a reduced quantity of MRP8/MRP14 heterodimers

(76) Inventors: Jorn-Peter Halle, Penzberg (DE); Andreas Goppelt, Munchen (DE)

Correspondence Address: CLARK & ELBING LLP, 101 FEDERAL STREET, BOSTON, MA, 02110, US

(21) Appl. No.: 10/134,841

(22) Filed: Apr. 29, 2002

Related U.S. Application Data
(60) Provisional application No. 60/322,925, filed on Sep. 17, 2001.

(30) Foreign Application Priority Data
Apr. 30, 2001 (DE) 10121254.2

(51) Int. Cl. C12Q 1/68; G01N 33/567

(52) U.S. Cl. 435/6; 435/7.21

(57) ABSTRACT

The present invention relates to the use of an MRP8/MRP14 heterodimer, or of its individual components in combination, of at least one nucleic acid encoding the entire heterodimer or its individual components in combination, or of a cell which is expressing the entire heterodimer, or its individual components in combination, for treating and/or preventing skin diseases, wounds, and/or wound-healing disturbances having a reduced quantity of MRP8/MRP14 heterodimers, in particular diabetes-associated wounds, and to methods for identifying pharmacologically active substances which exert an influence on the function or expression of MRP8/MRP14 heterodimers.

The present invention relates to the use of an MRP8/MRP14 heterodimer, or of its individual components in combination, of at least one nucleic acid encoding the entire heterodimer or its individual components in combination, or of a cell which is expressing the entire heterodimer, or its individual components in combination, for treating and/or preventing skin diseases, wounds, and/or wound-healing disturbances having a reduced quantity of MRP8/MRP14 heterodimers, in particular diabetes-associated wounds, and to methods for identifying pharmacologically active substances which exert an influence on the function or expression of MRP8/MRP14 heterodimers.

Skin wounds in healthy patients normally heal without any problems. However, a large number of temporal and spatial changes in the cell composition of the skin is required in order to achieve complete healing of the tissue. This process can last up to 2 years and, in non-fetal tissue, is always associated with scar formation. This points to the enormous complexity of the wound healing process in the skin. In wound healing, it is possible to distinguish the temporally consecutive, partially overlapping phases of coagulation, inflammation, proliferation and remodelling. Blood platelets, which release growth and coagulation factors, aggregate during coagulation. A fibrin matrix is formed, thus enabling cells to migrate within the wound. Subsequently, an inflammatory reaction develops approx. 5-7 days after the injury. In association with this, a variety of cell types, in particular neutrophilic granulocytes and monocytes, migrate into the wound and release mediators of the inflammatory reaction. The proliferation phase is required for restoring the blood vessels, regenerating damaged tissue and restructuring the regenerated tissue. The processes involved comprise, in particular, neovascularization, fibroblast proliferation and reepithelialization by means of the proliferation and differentiation of keratinocytes. The fibroblasts secrete several growth factors, such as PDGF and TGF-beta, which in turn regulate the synthesis and deposition of components of the extracellular matrix (ECM), such as fibronectin, laminin, glycosaminoglycans and collagen. During the reorganization of the tissue, the ECM components, particularly the collagen, are rearranged. As a result of collagen being continuously degraded and freshly synthesized, the reepithelialized wound can mature, and a flat scar is formed within 2 years. Once again, a large number of growth factors and chemoattractants are required for reconstructing the tissue in a coordinated manner. Thus, interleukin 1, TNF-beta and interferongamma exert an influence on secretion of the ECM components. TGF-beta, PDGF and FGF are also essential for the reorganization. Because of the large number of complex physiological processes which are involved in wound healing, a great variety of factors can cause disturbances of wound healing. These factors include, for example, ageing, immune system diseases, nutritional deficiencies, zinc deficiency, disturbances in innervation or blood flow, diabetes, alcohol abuse and genetic defects. Severe impairments in the wound healing process can in turn lead to chronic wounds and finally to ulcers. However, the therapies which have to date been developed for being able to intervene in chronic wound-healing disturbances offer little satisfaction. Established forms of therapy are restricted to physical support of the wound healing (e.g. dressings, compresses and gels), to scraping out the necrotic tissue, and to the transplantation of skin cells which have been cultured from skin tissues and/or of matrix proteins. While the therapeutic use of growth factors has been tried out for improving wound healing in recent years, it has not improved conventional therapy in any decisive manner; only PDGF-BB has been authorized for treating venous foot ulcers.

However, an aspect of wound-healing disturbances which has so far been disregarded are the pathogenic backgrounds which underlie the respective disturbances and which, because of their different molecular causes, require different therapies. Even though a number of wound-healing disturbances are distinguished clinically, molecular biological research is restricted to the investigation of venous ulcers, which are investigated as being representative of chronic wounds and/or wounds which heal poorly. The term chronic skin wounds normally covers very different diseases having different pathogenic backgrounds. In general, because they are the most frequent representatives, a distinction is made between diabetic ulcers, venous ulcers, arterial ulcers and decubitus ulcers. Decubitus ulcers are due to the continuous effect of pressure over long periods and are very deep wounds which are accompanied by necrosis, infection and maceration of the tissue. By contrast, venous ulcers, which are induced by venous stasis, are more superficial. On the other hand, arterial ulcers are frequently caused by arterial occlusion diseases. Diabetic ulcers, for their part, are ulcers which occur frequently in diabetes patients. In addition to a large number of diseases, the late complications of diabetes also encompass characteristic skin changes such as frequent infections, trophic disturbances and necrobiosis lipoidica. These changes can then, frequently as a result of microangiopathic disturbances, develop into poorly healing ulcers. The epidemiological importance of these diseases is made clear by the following statistical data. 25% of patients with type II diabetes frequently suffer from chronic ulcers (e.g. “diabetic foot”), about half of which require elaborate in-patient treatment and heal poorly. Diabetic foot on its own gives rise to more hospital admissions than any other complication associated with diabetes. The number of these cases associated with diabetes type I and type II is on the increase and represents approx. 2.5% of all hospital admissions.

The object of the present invention is therefore to find a novel active compound which decisively improves the healing and/or prevention of skin diseases, wounds and/or wound-healing disturbances having a reduced quantity of MRP8/MRP14 heterodimers, in particular of diabetes-associated wounds which heal poorly. Within the meaning of the present invention, “diabetes-associated wounds which heal poorly” are to be understood as being skin wounds in mammals and humans suffering from diabetes. Examples of such skin wounds are ulcers which are caused by diabetes, for example ulcus cruris arteriosum or necrobiosis lipoidica.

Surprisingly, it has now been found that the heterodimer consisting of the complexed polypeptides MRP8 and MRP14 (MRP8/MRP14) is expressed to a decreased extent particularly in diabetic wounds. Thus, the MRP8 mRNA is, for example, present at markedly lower concentrations in biopsies taken from a human diabetic ulcer than in biopsies taken from a venous ulcer or in biopsies taken from a normally healing wound. Furthermore, MRP14 polypeptide is virtually absent from the wound fluid taken from a diabetic patient whereas a strong MRP14 signal was observed, by means of immunostaining, in wound fluid taken from normally healing wounds and in venous ulcers. This is evidence that diabetes-associated wound-healing disturbances, in particular, are distinguished by a decreased abundance, which is specific for these diseases, in the expression of the MRP8 and MRP14 polypeptides and consequently of the heterodimer MRP8/MRP14. Furthermore, it was possible to demonstrate, by means of the experiments of the present invention, that compensating for the deficiency of MRP8/MRP14 in diabetic rabbits resulted in an extraordinary increase in the rate of wound healing. Similarly genetherapeutic treatment of diabetic rats with both MRP8 and MRP14 genes resulted in significantly improved wound healing. These results demonstrate that diabetes-associated wound-healing disturbances, in particular, can be successfully and effectively treated by increasing the quantity of MRP8/MRP14 nucleic acids or polypeptide in the wounds. In association with which the mechanism underlying diabetes-associated wound-healing disturbances is novel and the treatment in accordance with the invention can also be extended to all diseases having a deficiency of MRP8/MRP14 heterodimers.

Also, surprisingly, an assay for human MRP8/MRP14 heterodimer function on cells could be made available for the first time. Though human MRP8/MRP14 heterodimer is known for a long time, no effect of the human polypeptides on cellular activity could be determined. Thus, it was possible to demonstrate for the first time, that the human MRP8/MRP14 heterodimer exhibits a positive effect on the migration of keratinocytes, thus providing a cell-based assay to test substances for their influence on MRP8/MRP14 heterodimer function. The assay can be used to screen for pharmacologically active substances which can be used to treat and/or prevent diseases characterized by a disregulated amount or activity of human MRP8/MRP14 heterodimer or its individual components in combination. It can also be used for monitoring the activity of purified or recombinantly expressed MRP8/MRP14, e.g. in a quality assurance procedure.

The present invention therefore relates to the use of an MRP8/MRP14 heterodimer, or of its individual components in combination, of at least one nucleic acid encoding the entire heterodimer or its individual components in combination, or of a cell which is expressing the entire heterodimer, or its individual components in combination, for diagnosing, treating and/or preventing skin diseases, wounds and/or wound-healing disturbances having a reduced quantity of MRP8/MRP14 heterodimers, in particular diabetes-associated wounds. A preferred diabetes-associated wound which heals poorly is the diabetic ulcer.

The invention also relates to a functional assay on the activity of human MRP8/MRP14 heterodimer or its individual components in combination, or of at least one nucleic acid encoding the beterodimer or its individual components in combination, comprising the steps of:

1) bringing the human MRP8/MRP14 heterodimer or its individual components in combination, or the at least one nucleic acid encoding the entire heterodimer or its individual components in combination into contact with at least one cell,

2) treating the at least one cell with at least one test substance,

3) measuring migration of the at least one cell, and

4) comparing the measured migration of the at least one cell treated with the at least one test substance with the migration of at least one control cell which was/were not treated with the at least one test substance.

The object of the present invention is solved by the embodiments of the invention described in the claims. The claims are hereby incorporated by reference.

Preferably, the test substances exhibit a positive effect on migration. Such substances can be used to treat and/or prevent skin diseases, wounds and/or wound-healing disturbances having a reduced quantity of MRP8/MRP14 heterodimers, in particular diabetes-associated wounds. The invention also relates to a use of this assay for the identification of pharmacologically active substances.

MRP8 and MRP14 are polypeptides which have apparent molecular weights of 8 and 14 kDa respectively and which can form heterodimers in vivo, with both the monomeric and the heterodimeric forms being able to exhibit functional activities in mammals (see below). The monomers both belong to the family of the calgranulin or S100 polypeptides (Kliginann and Hilt, Trends Biochem. Sci., 13: 437-447). The polypeptides of these families are Ca²⁺-binding and Ca²⁺-modulated polypeptides which form antiparallel, noncovalent dimers and which regulate a very wide variety of cellular functions such as cell growth, differentiation, energy metabolism and cytoskeleton-membrane interactions.

The MRP8 and MRP14 polypeptides are principally expressed in neutrophilic granulocytes and monocytes and also in activated macrophages (Hessian et al., 1993, J. Leukocyte Biol., 53: 197-204). Initial studies suggested that, in humans, the heterodimer MRP8/MRP14 acts as a factor inhibiting macrophage migration (Burmeister et al., 1986, Immunbiol., 171: 461-474; EP 0162 812), a finding which it has not, however, been possible to confirm. To date, it has not been possible to demonstrate that the heterodimer or the monomers have any chemotactic activity (Hessian et al., see above). On the other hand, the murine homolog of MRP8 appears on its own to act as a powerful chemoattractant for neutrophilic granulocytes (Lackrnann et al., 1992, J. Biol. Chem., 267: 7499-7504). By contrast, it has been suggested that the human MRP8/MRP14 heterodimer plays a role in the adhesion of leukocyte cells to vascular endothelium (Newton and Hogg; 1998, J. Immunol., 160: 1427-1435). In addition, MRP8 and MRP14 are expressed in keratinocytes which developed abnormally in cell culture (Olsen et al., 1995, Electrophoresis, 16: 2241-2248). Examples of biochemically characterized functions of the human MRP8/MRP14 heterodimer are those of binding fatty acid and calcium, with the calcium binding exerting an influence on the fatty acid binding (Kerkhoffet al., 1999, J. Biol. Chem., 274: 32672-9).

The following section shows that, while a number of publications have related to MRP8 or MRP14 with diseases which are generally characterized by an increased quantity of MRP8 and MRP14, MRP8 and MRP14 have not been related to the treatment and/or prevention of skin diseases, wounds and/or wound-healing disturbances having a reduced quantity of MRP8/MRP14 heterodimers, in particular diabetes-associated wounds which heal poorly. It is therefore suprising that an MRP8/MRP14 heterodimer, or its individual components in combination, at least one nucleic acid encoding the entire heterodimer or its individual components in combination, or a cell which is expressing the entire heterodimer, or its individual components in combination, can be used for treating and/or preventing skin diseases, wounds and/or wound-healing disturbances having a reduced quantity of MRP8/MRP14 heterodimers, in particular diabetes-associated wounds.

The quantity of MRP8 and MRP14 is greatly increased in association with inflammatory diseases, such as rheumatoid arthritis, inflammatory intestinal diseases, psoriasis, lung inflammation and the rejection of foreign implants (Brandtzaeg et al., 1987, Am. J. Clin. Pathol., 87: 700-707; Wilkinson et al., 1988, J. Cell Science, 91: 221-230; Kelly et al., 1991, Br. J. Dermatol., 124: 403-409; Madsen et al., 1992, J. Invest. Dermatol., 99: 299-305; Roth et al., 1992, Int. Arch. Allergy. Immunol., 98: 140-145; Kunz et al., 1992, Arch. Dermatol. Res, 284: 386-390). Further investigations confirm the role of MRP8 and/or MRP14 in delayed allergic hypersensitivity reactions of the skin and in skin disturbances such as atopic dermatitis and psoriasis and in association with granulomatoses (Hardas et al., 1996, J. Invest. Dermatol., 106: 753-758; Lackmann et al., 1992, J. Biol. Chem., 267: 7499-7504; WO 92/04376). In humans, the expression of MRP8 and MRP14 by phagocytes is increased in association with contact dermatitis (Frantzen et al., 1993, Int. Arch. Dermatol. Immunol., 101: 182-189; Roth et al., 1992, Int. Arch. Dermatol. Immunol., 98: 140-145). Furthermore, the heterodimer has been repeatedly detected in the epidermis of patients suffering from lichen planus, lupus erythematosus and psoriasis vulgaris but not, however, in the epidermis of normal skin or in patients suffering from leukocytoclastic vasculitis (Kunz et al., 1992, Arch Dermatol. Res., 284: 386-390). MRP14 is strongly expressed in mononuclear phagocytes in granulomatous diseases whereas MRP8 is expressed in granuloma of the foreign body type, erythema nodosum and cat-scratch disease but not, or only weakly, in phagocytes in association with sarcoidosis and tuberculosis. While labeling with an antibody against MRP8/MRP14 was observed in suprabasal keratinocytes in the skin of patients suffering from chronic discoid lupus erythematosus, it was not possible to detect any labeling in the case of patients suffering from Jessner's lymphocytic infiltration of the skin (Kunz et al., 1999, Eur. J. Dermatol., 9: 107-110).

Within the meaning of the present invention, “MRP8/MRP14 heterodimers” are dimers which are associated by way of noncovalent bonds and which contain a mouse or human MRP8 polypeptide as depicted in SEQ ID No. 1 or 2, or an active functional variant thereof, and a mouse or human MRP14 polypeptide as depicted in SEQ ID No. 3 or 4, or a functional variant thereof.

Within the meaning of the invention, “individual components” of the MRP8/MRP14 heterodimer are mouse or human MRP8 polypeptides as depicted in SEQ ID No. 1 to 2, or functional variants thereof, or nucleic acids encoding them, or variants thereof, and mouse or human MRP14 polypeptides as depicted in SEQ ID No. 3 to 4, or functional variants thereof, or nucleic acids encoding them, or variants thereof.

Within the meaning of the present invention, the term “functional variants” is to be understood as denoting MRP8 polypeptides or MRP14 polypeptides which form stable heterodimers with an MRP14 polypeptide or an MRP8 polypeptide, respectively. The formation of the heterodimer can be determined, for example, by means of mass spectrometry (Strupat et al., 2000, J. Am. Soc. Mass Spectrom., 11: 780-788). For example, variants of said polypeptides possess at least approximately 70%, in particular at least approximately 80%, especially at least approximately 90%, sequence identity with one of the sequences SEQ ID No. 1 to SEQ ID No. 4. Functional variants of the polypeptide can also be parts of the polypeptides used in accordance with the invention provided the function of the polypeptide is not significantly altered. Such amino acids can, for example, be identified by means of alanine scanning (see, e.g., Nagashima et al., 1993, J. Biol. Chem., 268: 2888-92). Examples of such functional variants are the polypeptides which are homologous to the polypeptides which can be used in accordance with the invention and which are derived, in particular, from organisms other than humans or mice, preferably from nonhuman mammals such as monkeys, pigs and rats. Other examples are polypeptides which are encoded by different alleles of the gene, in different individuals or in different organs of an organism. Furthermore, a posttranslational or cotranslational modification of the polypeptide chain which is present in the native state can be lacking or be altered without this alteration signficantly impairing the activity of the polypeptides. The invention also encompasses N-terminal and/or C-terminal, and/or internal, deletions of the polypeptide in the range of approx. 1-15, preferably of approx. 1-10, in particular of approx. 1-5, amino acids. For example, the first amino acid, i.e. methionine, can be missing without the function of the polypeptide being significantly altered.

In order to decide, whether a candidate polypeptide is a functional variant, the activity of the candidate functional variant polypeptide may be compared with the activity of a polypeptide according to the invention. Assuming that the candidate functional variant polypeptide fulfills the criteria of a functional variant on the level of % sequence identity the candidate functional variant molecule represents a functional variant if the activity in the functional assays is similar to or identical with the activity exhibited by the polypeptide useable according to the invention.

Such standard wound healing assays comprise for example the application of an expression vector containing at least one nucleic acid coding for the candidate polypeptide heterodimer or its components or the application of the candidate polypeptide heterodimer or its components itself to wounds. After incubation of, for example an expression vector, the progress of wound healing of wounds that have been injected with different expression vectors containing either the nucleic acid coding for the candidate functional variant polypeptide(s) or the nucleic acid coding for the polypeptide according to the invention is compared. Such assays may also be applied to test the activity of candidate functional variant polypeptides in the case of various wound healing models, for example badly healing wounds of dexamethasone-treated animals. For example, it was demonstrated that application of the polypeptide-variants PDGF-A and PDGF-B on badly healing rabbit wounds resulted in a comparable wound healing response (J. Surg. Res., 2000, 93:230-236).

An alternative test for functional variants is a migration assay for keratinocytes, for example as described in Example 4. Thus, a migration assay comprises for example the application of an expression vector containing at least one nucleic acid coding for the candidate polypeptide heterodimer or its components or the application of the candidate polypeptide heterodimer or its components itself to keratinocytes. After incubation of, for example an expression vector, the migration of keratinocytes that have been transfected with different expression vectors containing either the nucleic acid coding for the candidate functional variant polypeptide(s) or the nucleic acid coding for the polypeptide according to the invention is compared. Migration assays suitable for keratinocytes are known to the skilled person and comprise for example, the Boyden chamber assay, the colloidal gold assay, the scratch assay and an assay based on the migration in a fibrin matrix, which are described below in detail.

Within the meaning of the invention the function of the MRP8/MRP14 heterodimer or the individual components of the MRP8/MRP14 heterodimer in combination is understood to encompass the activity of the heterodimer or of its individual components in combination, i.e. especially the activity the heterodimer or of its individual components in combination exert onto the migration of cells, especially onto skin cells such as keratinocytes. The activity of the MRP8/MRP14 heterodimer or of its individual components in combination further encompass the chemotrophic acitvity, cell-adhesion, binding activity with respect to fatty acid and calcium, influence onto cell growth, differentiation, energy metabolism and cytoskeleton-membrane interactions.

The term “coding nucleic acid” relates to RNA or DNA which encodes a polypeptide which can be used in accordance with the invention or its functional variants or a precursor stage, for example a propolypeptide or prepropolypeptide, thereof.

The term “variants” denotes all the DNA sequences which are complementary to a DNA sequence (reference sequence), which encode polypeptides which can be used in accordance with the invention and which have the sequences depicted in SEQ ID No. 1 to SEQ ID No. 4, or their functional variants, and which exhibit at least approx. 70%, in particular at least approx. 80%, especially at least approx. 90%, sequence homology with the reference sequence. The term “variants” furthermore designates all the DNA sequences which are complementary to the reference sequence and hybridize with it under stringent conditions and which encode a polypeptide which exhibits essentially the same activity as that of the polypeptide encoded by the reference sequence and also their degenerate forms.

Sequence identity is understood as degree of identity (% identity) of two sequences, that in the case of polypeptides can be determined by means of for example BlastP 2.0.1 and in the case of nucleic acids by means of for example BLASTN 2.014, wherein the Filter is set off and BLOSUM is 62 (Altschul et al., 1997, Nucleic Acids Res., 25:3389-3402). “Sequence homology” is understood as similarity (% positives) of two polypeptide sequences determined by means of for example BlastP 2.0.1 wherein the Filter is set off and BLOSUM is 62 (Altschul et al., 1997, Nucleic Acids Res., 25:3389-3402).

It is known that changes in the sequence of the nucleic acids which can be used in accordance with the invention can be present, for example as a result of the degeneracy of the genetic code, or that untranslated sequences can be present at the 5′ end and/or the 3′ end of the nucleic acid, without the activity of the encoded polypeptide being significantly altered. This invention therefore also encompasses so-called “variants” of the previously described nucleic acids.

The term “stringent hybridization conditions” is to be understood, in particular, as meaning those conditions in which a hybridization takes place, for example, at 60° C. in 2.5×SSC buffer followed by several washing steps at 37° C. in a lower buffer concentration and remains stable.

The nucleic acids encoding MRP8 and MRP14 polypeptides which can be used in accordance with the invention are preferably DNA or RNA, preferably DNA, in particular double-stranded DNA. Furthermore, the sequence of the nucleic acids can be characterized by the fact that it possesses at least one intron and/or a polyA sequence.

In general, a double-stranded DNA is preferred for expressing the relevant genes, both for preparing the polypeptides which can be used in accordance with the invention and in association with vectors which can be used in accordance with the invention and which are applicable in gene therapy, with the DNA region encoding the polypeptide being particularly preferred. In eucaryotes, this region begins with the first start codon (ATG) which is located in a Kozak sequence (Kozak, 1987, Nucleic. Acids Res. 15: 8125-48) and extends to the next stop codon (TAG, TGA or TAA) which is located in the same reading frame as the ATG. In the case of procaryotes, this region begins with the first AUG (or GUG) after a Shine-Dalgamo sequence and ends with the next stop codon (TAG, TGA or TAA) which is located in the same reading frame as the ATG.

Furthermore, nucleic acids which have been prepared synthetically can be used according to the invention. Thus, the nucleic acids which are used in accordance with the invention can, for example, be synthetisized chemically, e.g. according to the phosphotriester method, with the aid of the DNA sequences described in Table 1 and/or with the aid of the polypeptide sequences which are likewise described in this table by referring to the genetic code (see, e.g., Uhlmann, E. & Peyman, A. (1990) Chemical Reviews, 90, 543-584, No. 4).

The polypeptides which can be used in accordance with the invention can also be prepared synthetically. Thus, the entire polypeptide, or parts thereof, can, for example, be synthesized by classical synthesis (Merrifield technique). Particularly preferred is the use of polypeptides which have been prepared recombinantly using one of the previously described nucleic acids. Furthermore, MRP8 and MRP14 polypeptides can be isolated from an organism or from tissue or cells and used in accordance with the invention. Thus it is possible, for example, to purify polypeptides which can be used in accordance with the invention from granulocytes (van den Bos et al., 1998, Protein Expr. Purif., 13: 313-318). Furthermore, cell lines can be prepared from MRP8 and/or MRP14-expressing cells and then used for isolating MRP8 and/or MRP14.

In a preferred embodiment of the invention, the wounds are diabetes-associated wounds, in particular a diabetic ulcer. Furthermore, the wounds are preferably of large surface area.

In another embodiment of the invention, at least one nucleic acid which can be used in accordance with the invention and which encodes an MRP8 polypeptide is contained in an expression cassette in a vector, preferably in an expression vector or in a vector which is applicable in gene therapy, and at least one nucleic acid which can be used in accordance with the invention and which encodes an MRP14 polypeptide is contained in another expression cassette in another vector, preferably in an expression vector or a vector which is applicable in gene therapy. These vectors which can be used in accordance with the invention can then be employed, in combination, as drugs for treating and/or preventing skin diseases, wounds and/or wound-healing disturbances having a reduced quantity of MRP8/MRP14 heterodimers, in particular diabetes-associated wounds which heal poorly (see Examples 3, 6). The vectors which are applicable in gene therapy preferably contain wound-specific, skin-specific or constitutively active regulatory sequences which are functionally connected to the previously described nucleic acids.

In a preferred embodiment of the invention, at least one nucleic acid which can be used in accordance with the invention and which encodes an MRP8 polypeptide is contained, together with a nucleic acid which can be used in accordance with the invention and which encodes an MRP14 polypeptide, in an expression cassette in a vector, preferably in an expression vector or in a vector which is applicable in gene therapy (see, e.g. Example 3). These vectors which can be used in accordance with the invention can then be employed as drugs for treating and/or preventing skin diseases, wounds and/or wound-healing disturbances having a reduced quantity of MRP8/MRP14 heterodimers, in particular diabetes-associated wounds which heal poorly. The vectors which are applicable in gene therapy preferably contain wound-specific, skin-specific or constitutively active regulatory sequences which are linked functionally to the previously described nucleic acids.

The expression vectors which are used for preparing a polypeptide which can be used in accordance with the invention can be prokaryotic or eukaryotic expression vectors. Examples of prokaryotic expression vectors are the pGEM vectors or pUC derivatives, which are used for expression in E. coli, and examples of eukaryotic expression vectors are the vectors p426Met25 or p426GAL1 (umberg et al. (1994) Nucl. Acids Res., 22, 5767-5768), which are used for expression in Saccharomyces cerevisiae, the Baculovirus vectors, as disclosed in EP-B1-0 127 839 or EP-B1-0 549 721, which are used for expression in insect cells, and the vectors Rc/CMV and Rc/RSV, or SV40 vectors, which are used for expression in mammalian cells, with all these vectors being generally available.

In general, the expression vectors also contain promoters which are suitable for the respective cell, such as the trp promoter for expression in E.coli (see, e.g., EP-B1-0 154 133), the Met 25, GAL 1 or ADH2 promoter for expression in yeasts (Russel et al. (1983), J. Biol. Chem. 258, 2674-2682; Mumberg, see above), and the baculovirus polyhedrin promoter for expression in insect cells (see, e.g., EP-B1-0 127 839). Promoters which permit constitutive, regulatable, tissue-specific, cell type-specific, cell cycle-specific or metabolism-specific expression in eukaryotic cells are suitable, for example, for expression in mammalian cells. Regulatable elements in accordance with the present invention are promoters, activator sequences, enhancers, silencers and/or repressor sequences.

Examples of preferred regulatable elements which permit constitutive expression in eucaryotes are promoters which are recognized by RNA polymerase III or viral promoters, CMV enhancer, CMV promoter (see also Example 3), SV40 promoter or LTR promoters, e.g. derived from MMTV (mouse mammary tumor virus; Lee et al. (1981) Nature 214, 228-232) and other viral promoter and activator sequences which are derived from, for example, HBV, HCV, HSV, HPV, EBV, HTLV or HIV.

Examples of regulatable elements which permit inducible expression in eucaryotes are the tetracycline operator in combination with an appropriate repressor (Gossen M. et al. (1994) Curr. Opin. Biotechnol. 5, 516-20).

The expression of nucleic acids which can be used in accordance with the invention preferably takes place under the control of tissue-specific promoters, with skin-specific promoters, such as the human K10 promoter (Bailleul et al., 1990. Cell 62: 697-708), the human K14 promoter (Vassar et al., 1989, Proc. Natl. Acad. Sci. USA 86: 1563-67) or the bovine cytokeratin IV promoter (Fuchs et al., 1988; The Biology of Wool and Hair (eds.: G. E. Rogers, et al.), pp. 287-309. Chapman and Hall, London/New York) being particularly to be preferred.

Other examples of regulatable elements which permit tissue-specific expression in eucaryotes are promoters or activator sequences from promoters or enhancers of those genes which encode polypeptides which are only expressed in particular cell types.

Examples of regulatable elements which permit cell cycle-specific expression in eucaryotes are promoters of the following genes: cdc25, cyclin A, cyclin E, cdc2, E2F, B-myb or DHFR (Zwicker J. and Müiller R. (1997) Trends Genet. 13, 3-6).

An example of a regulatable element which permits keratinocyte-specific expression in skin is the FiRE element (Jaakkola et al., 2000, Gen. Ther., 7: 1640-1647). The FiRE element is an AP-1-driven, FGF-inducible response element of the syndecan-1 gene (Jaakkola et al., 1998, FASEB J., 12: 959-9).

Examples of regulatable elements which permit metabolism-specific expression in eucaryotes are promoters which are regulated by hypoxia, by glucose deficiency, by phosphate concentration or by heat shock.

In order to enable the nucleic acids used in accordance with the invention to be introduced into a eukaryotic or prokaryotic cell by means of transfection, transformation or infection, and thereby to enable the polypeptide to be expressed, the nucleic acid can be present as a plasmid, or as a part of a viral or non viral vector. Particularly suitable viral vectors in this connection are: baculoviruses, vaccinia viruses, adenoviruses, adeno-associated viruses and herpesviruses. Particularly suitable non viral vectors are for example: virosomes, liposomes, cationic lipids and polylysine-conjugated DNA.

Examples of vectors which are applicable in gene therapy are viral vectors, for example adenoviral vectors or retroviral vectors (see Example 6; Lindemann et al., 1997, Mol. Med. 3: 466-76; Springer et al., 1998, Mol. Cell. 2: 549-58). Eukaryotic expression vectors are suitable for use in gene therapy when present in isolated form since naked DNA can penetrate into skin cells when applied topically (Hengge et al., 1996, J. Clin. Invest. 97: 2911-6; Yu et al., 1999, J. Invest. Dermatol. 112: 370-5).

Vectors which are applicable in gene therapy can also be obtained by complexing the nucleic acid used in accordance with the invention with liposomes, since this makes it possible to achieve a very high efficiency of transfection, particularly of skin cells (Alexander and Akhurst, 1995, Hum. Mol. Genet. 4: 2279-85). In lipofection, small, unilamellar vesicles consisting of cationic lipids are prepared by subjecting the liposome suspension to ultrasonication. The DNA is bound ionically on the surface of the liposomes, specifically in a relationship which is such that a positive net charge remains and 100% of the plasmid DNA is complexed by the liposomes. In addition to the DOTMA (1,2-dioloyloxypropyl-3-trimethylammonium bromide) and DPOE (dioleoylphosphatidylethanolamine) lipid mixtures employed by Felgner et al. (1987, see above), a large number of new lipid formulations have by now been synthesized and tested for their efficiency in the transfection of various cell lines (Behr, J. P. et al. (1989), Proc. Natl. Acad. Sci. USA 86, 6982-6986; Felgner, J. H. et al. (1994) J. Biol. Chem. 269, 2550-2561; Gao, X. & Huang, L. (1991), Biochim. Biophys. Acta 1189, 195-203). Examples of the new lipid formulations are DOTAP N-[1-(2,3-dioleoyloxy)propyl]-N,N,N-trimethylammonium ethyl sulfate or DOGS (TRANSFECTAM; dioctadecylamidoglycylspermine). The Cytofectin GS 2888 cationic lipids have also proved to be very well suited for transfecting keratinocytes in vitro and in vivo (U.S. Pat. No. 5,777,153; Lewis et al., 1996, Proc. Natl. Acad. Sci. USA, 93: 3176-3181). Auxiliary substances which increase the transfer of nucleic acids into the cell can, for example, be proteins or peptides which are bound to DNA or synthetic peptide-DNA molecules which make it possible to transport the nucleic acid into the nucleus of the cell (Schwartz et al. (1999) Gene Therapy 6, 282; Brandén et al. (1999) Nature Biotech. 17, 784). Auxiliary substances also encompass molecules which enable nucleic acids to be released into the cytoplasm of the cell (Planck et al. (1994) J. Biol. Chem. 269, 12918; Kichler et al. (1997) Bioconj. Chem. 8, 213). Liposomes are a pharmaceutically acceptable carrier within the meaning of the present invention. Liposomes comprise multilamellar vesicles (MLVs), small unilamellar vesicles (SLTs) and large unilammellar vesicles (LUVs).

Methods for preparing liposome-nucleic acid complexes are known to the skilled person (e.g. Straubinger et al., 1983, in Methods of Immunology, 101: 512-527; Szoka et al., 1978, Proc. Natl. Acad. Sci. USA, 75: 4194-4198). The term “liposomes” encompasses, for example, liposomal compositions which are disclosed in U.S. Pat. No. 5,422,120, WO 95/13796, WO 94/23697, WO 91/14445 and EP 524,968 B1. Liposomes can be used as a pharmaceutical carrier for either or both of the nucleic acids which can be used in accordance with the invention and the polypeptides which can be used in accordance with the invention; they are preferably used as a pharmaceutical carrier for the nucleic acids which can be used in accordance with the invention. The therapeutically active substance can be conjugated to the liposome or it can be conjugated to a hydrogel polymer, with it being possible for the hydrogel polymer (or a component of the hydrogel polymer) to be conjugated to a liposome or to be enclosed by a liposome. Another particularly suitable form of vectors for gene therapy can be obtained by applying the nucleic acid used in accordance with the invention to gold particles and using for example a Gene Gun to administer the charged particles topically by firing them into the skin or cells (Example 3; Wang et al., 1999, J. Invest. Dermatol., 112:775-81, Tuting et al., 1998, J. Invest. Dermatol. 111:183-8). Devices for performing intradermal injection using pressure have been disclosed, for example, in U.S. Pat. No. 5,630,796.

Another form of vector which is applicable in gene therapy can be prepared by introducing “naked” expression vectors into a biocompatible matrix, for example a collagen matrix. This matrix can, for example, be introduced into diabetes-associated wounds in order to transfect the immigrating cells with the expression vector and to express the polypeptides used in accordance with the invention in the cells (Goldstein and Banadio, U.S. Pat. No. 5,962,427).

For the use of the previously described nucleic acid in gene therapy it is also advantageous if the part of the nucleic acid which encodes the polypeptide contains one or more noncoding sequences, including intron sequences, preferably between the promoter and the start codon for the polypeptide (see Example 3) and/or a polyA sequence, in particular the naturally occurring polyA sequence or an SV40 virus polyA sequence, in particular at the 3′ end of the gene since this makes it possible to stabilize the mRNA (Jackson, R. J. (1993) Cell 74, 9-14 and Palmiter, R. D. et al. (1991) Proc. Natl. Acad. Sci.USA 88, 478-482).

Cells can be either prokaryotic or eukaryotic cells, examples of prokaryotic cells are E. coli, and examples of eukaryotic cells are Saccharomyces cerevisiae or insect cells. Thus, E. coli BL21 cells have, for example, proved to be suitable cells for expressing human MRP8 and MRP14 (Hunter and Chazin, 1998, J. Biol. Chem., 273: 12427-12435). The use of E. coli cells for preparing polypeptides which can be used in accordance with the invention constitutes a preferred embodiment. The polypeptides which can be used in accordance with the invention are prepared, for example, by expressing the previously described nucleic acid in a suitable expression system, as already described above, using methods which are well known to the skilled person. Examples of suitable cells are the E. coli strains DHS, HB101 or BL21, the yeast strain Saccharomyces cerevisiae, the insect cell line Lepidopteran, e.g. from Spodoptera frugiperda, or the animal cells COS, Vero, 293, HaCaT and HeLa, all of which are generally available.

The invention furthermore relates to the use of an MRP8/MRP14 heterodimer, or of its individual components in combination, with at least one individual component being employed in the form of a fusion protein for treating and/or preventing skin diseases, wounds and/or wound-healing disturbances having a reduced quantity of MRP8/MRP14 heterodimers, in particular of diabetes-associated wounds which heal poorly, with the fusion protein being prepared using a previously described nucleic acid.

This involves preparing fusion proteins which contain the above-described MRP8 and/or MRP14 polypeptides, with the fusion proteins themselves already being active or only becoming active after the fusion moiety has been eliminated. These fusion proteins include, in particular, fusion proteins having a content of approx. 1-300, preferably approx. 1-200, particularly preferably approx. 1-150, in particular approx. 1-100, especially approx. 1-50 foreign amino acids. Examples of such peptide sequences are prokaryotic peptide sequences which can be derived, for example, from E. coli galactosidase.

Other preferred examples of peptide sequences for fusion proteins are peptides which facilitate detection of the fusion protein; examples of these are the green fluorescent protein or functional variants thereof. Thus, murine MRP14 has already been expressed as a fusion protein in mammalian cells (Nacken et al., 2000, Eur. J. Biochem. 267: 560-565).

It is possible to add on at least one further “polypeptide tag” for the purpose of purifying the previously described proteins. For example, suitable protein tags enable the proteins which are to be purified to be absorbed with high affinity to a matrix. This is then followed, for example, by the following steps: stringent washing with suitable buffers without eluting the complex to any significant extent, and, subsequently, specific elution of the absorbed complex. Examples of the protein tags which are known to the skilled person are a (His)₆ tag, a Myc tag, a FLAG tag, a hemagglutinin tag, a glutathione transferase (GST) tag, intein having an affinity chitin-binding tag and a maltose-binding protein (MBP) tag. These protein tags can be located N-terminally, C-terminally and/or internally.

The present invention also relates to the use of an MRP8/MRP14 heterodimer, or of its individual components in combination, or of at least one nucleic acid encoding the entire heterodimer or its individual components in combination, or of a cell which is expressing the entire heterodimer or its individual components in combination, where appropriate combined with suitable additives and auxiliary substances, for producing a drug for treating and/or preventing skin diseases, wounds and/or wound-healing disturbances having a reduced quantity of MRP8/MRP14 heterodimers and/or diabetes-associated wounds, in which drug at least one MRP8 polypeptide as depicted in SEQ ID No. 1 or 2, or a functional variant thereof, or nucleic acids encoding them, or a variant thereof, and/or at least one MRP14 polypeptide as depicted in SEQ ID No. 3 or 4, or a functional variant thereof, or nucleic acids encoding them, or a variant thereof, or at least one cell expressing the entire heterodimer, or its individual components in combination is/are used, where appropriate together with suitable additives and auxiliary substances and/or carrier systems.

The therapy of skin diseases, wounds and/or wound-healing disturbances having a reduced quantity of MRP8/MRP14 heterodimers, in particular of diabetes-associated wounds, can be effected in a conventional manner, for example using dressings, plasters, compresses or gels which contain the drugs according to the invention. Thus, it is possible to administer drugs comprising suitable additives or auxiliary substances, such as physiological sodium chloride solution, demineralized water, stabilizer, proteinase inhibitors, gel formulations, such as white vaseline, low-viscosity paraffin and/or yellow wax, etc., topically and locally in order to exert an immediate and direct effect on the wound healing process. The topical administration of therapeutic compositions can be effected, for example, in the form of a cream, a foam, an aerosol spray, an injection, a gel matrix or a sponge or in the form of drops or washings. These topical forms of administration are preferred for the use of an MRP8/MRP14 heterodimer which can be used in accordance with the invention or of its individual components in combination. Furthermore, the drugs according to the invention can, where appropriate, be administered in the form of liposome complexes or gold particle complexes, likewise topically and locally in the region of the wound. This form of administration is preferred for vectors which are applicable in gene therapy and which contain at least one nucleic acid which can be used in accordance with the invention. The treatment can also be effected using a transdermal therapeutic system (TTS) which enables the drugs according to the invention to be released in a chronologically controlled manner. TTS have been disclosed, for example, in EP 0 944 398 A1, EP 0 916 336 A1, EP 0 889 723 A1 or EP 0 852 493 A1. However, the treatment with the drugs according to the invention can also be effected using oral dosage forms, such as tablets or capsules, by way of the mucous membranes, for example the nose or the oral cavity, or in the form of depots which are implanted under the skin.

Preference is given to using an MRP8/MRP14 heterodimer, or its individual components in combination, for producing a drug. Preference is given to drugs which bring about an increase in the quantity of both MRP8 polypeptides as depicted in SEQ ID No. 1 or 2, or functional variants thereof, and MRP14 polypeptides as depicted in SEQ ID No. 3 or 4, or functional variants thereof, in the wound fluid of the wound being treated. The polypeptides can be prepared synthetically or recombinantly or can be isolated from tissue or mammalian fluids, for example blood, with particular preference being given to preparation using one of the above-described expression systems, in particular E. coli cells. The recombinant polypeptides which have been prepared in this way can also be present as fusion proteins, e.g. for facilitating purification or detection.

Within the meaning of the present invention, “wound fluid” is understood as being the extracellular fluid in a wound, which fluid essentially contains no cells or cell debris. It is isolated by, for example, aspirating fluid out of a vacuum-sealed wound and subsequently centrifuging this fluid at approx. 200×g (4° C.; 15 min), decanting the supernatant and centrifuging the fluid once again (3300×g; 4° C.; 15 min).

Another preferred embodiment is the use of at least one nucleic acid encoding an MRP8 polypeptide as depicted in SEQ ID No. 1 or 2, or a variant thereof, and/or of at least one nucleic acid encoding an MRP14 polypeptide as depicted in SEQ ID No. 3 or 4, or a variant thereof, in combination, for treating and/or preventing skin diseases, wounds and/or wound-healing disturbances having a reduced quantity of MRP8/MRP14 heterodimers, in particular diabetes-associated wounds which heal poorly. The nucleic acids are preferably in the form of DNA, in particular in the form of double-stranded DNA. Particular preference is given to nucleic acids which possess at least one intron and/or a polyA sequence. These nucleic acids which can be used in accordance with the invention can then be introduced into the wound combined, as explained above, preferably as a constituent of expression vectors or of vectors which are applicable in gene therapy. In this connection, the nucleic acid encoding an MRP8 polypeptide as depicted in SEQ ID No. 1 or 2, or a variant thereof, and the nucleic acid encoding an MRP14 polypeptide as depicted in SEQ ID No. 3 or 4, or a variant thereof, can either be present together in a vector or be present in different vectors (see Example 3). If the two nucleic acids of the drug are present in different vectors, they can then be employed in the therapy simultaneously (see, e.g., Example 3) or at different time points and spatially separate or together.

In another preferred embodiment, use is made of cells which contain at least one nucleic acid encoding an MRP8 polypeptide as depicted in SEQ ID No. 1 or 2, or a variant thereof, and/or a cell containing at least one nucleic acid encoding an MRP14 polypeptide as depicted in SEQ ID No. 3 or 4, or a variant thereof, in combination, for producing a drug for treating and/or preventing skin diseases, wounds and/or wound-healing disturbances having a reduced quantity of MRP8/MRP14 heterodimers, in particular diabetes-associated wounds.

Preference is also given to using at least one cell, wherein the cell contains at least one nucleic acid encoding an MRP8 polypeptide as depicted in SEQ ID No. 1 or 2, or a variant thereof, together with a nucleic acid containing at least one nucleic acid encoding an MRP14 polypeptide as depicted in SEQ ID No. 3 or 4, or a variant thereof, for producing a drug for treating and/or preventing skin diseases, wounds and/or wound-healing disturbances having a reduced quantity of MRP8/MRP14 heterodimers, in particular diabetes-associated wounds.

Particular preference is given to cells which can be used in accordance with the invention and which contain the nucleic acids in the form of an above-described expression vector or vector which is applicable in gene therapy. The cells can then be introduced into the wound either directly or, where appropriate, combined with suitable carrier systems and/or additives and/or auxiliary substances. Suitable carrier systems have been disclosed, for example, in U.S. Pat. No. 5,980,888, WO 92/06179, EP 0242 270 or WO 90/02796. Preferred cells are autologous or allogenic skin cells, in particular keratinocytes, fibroblasts and endothelial cells. If the two nucleic acids are contained in different cells, they can then be employed in the therapy simultaneously or at different time points and spatially separate or together.

Particular preference is given to drugs which comprise nucleic acids which can be used in accordance with the invention and which are present in a vector or a cell, as explained above, or polypeptides which can be used in accordance with the invention, for treatment by means of gene therapy.

Another preferred transformed cell, which can be used in accordance with the invention, is a transgenic, embryonic, nonhuman stem cell wherein said transformed cell comprises one or more expression cassettes according to the invention. Methods for transforming cells and/or stem cells are well known to the skilled person and include, for example, electroporation and microinjection. The invention furthermore relates to the use of a transgenic, nonhuman mammal whose genome contains a previously-described expression cassette. In general, transgenic animals exhibit an elevated tissue-specific expression of the nucleic acids and/or polypeptides and are therefore suitable for preparing polypeptides which can be used in accordance with the invention. If, for example, a mammary gland-specific promoter is selected, the recombinant polypeptides which can be used in accordance with the invention can then be isolated from the milk which is produced (Clark, 1998, J. Mammary Gland Biol. Neoplasia, 3: 337-350). For example, expression of blood coagulation factor VIII in the mammary glands of transgenic sheep, under the control of the beta-lactoglobulin gene promoter, has been described (Niemann et al., 1999, Transgenic Res., 8: 237-247). It is furthermore possible to use nonembryonic eukaryotic cells in a mammal in accordance with the invention by providing, for example by means of transfection, suitable cells or organs with an expression vector which contains the nucleic acids which can be used in accordance with the invention. Thus, using DEAE dextran or polyionic complexes to transfect the guinea pig lactiferous duct with an expression vector containing the hGH gene results, for example, in continuous expression of hGH (Hens et al., Biochem. Biophys. Acta, 2000, 1523: 161-171).

Methods for preparing transgenic animals, in particular the mouse, are likewise known to the Ad skilled person from DE 196 25 049 and U.S. Pat. Nos. 4,736,866; 5,625,122; 5,698,765; 5,583,278 and 5,750,825 and comprise transgenic animals which can be generated, for example, by the direct injection of expression vectors (see above) into embryos or spermatocytes or by the transfection of expression vectors into embryonic stem cells (Polites and Pinkert: DNA Microinjection and Transgenic Animal Production, pages 15 to 68 in Pinkert, 1994: Transgenic Animal Technology: A Laboratory Handbook, Academic Press, London, UK: Houdebine, 1997, Harwood Academic Publishers, Amsterdam, The Netherlands; Doetschman: Gene Transfer in Embryonic Stem Cells, pages 115 to 146 in Pinkert, 1994, see above; Wood: Retrovirus-Mediated Gene Transfer, pages 147 to 176 in Pinkert, 1994, see above; Monastersky: Gene Transfer Technology; Alternative Techniques and Applications, pages 177 to 220 in Pinkert, 1994, see above).

The invention furthermore relates to the use of an MRP8/MRP14 heterodimer, or of its individual components in combination, of at least one nucleic acid encoding the entire heterodimer or its individual components in combination, or of a cell which is expressing the entire heterodimer or its individual components in combination, for preparing a diagnostic agent for diagnosing diabe-tes-associated wounds. In this context, the diagnostic agent contains at least one antibody which is directed against an MRP8/MRP14 heterodimer, against an MRP8 polypeptide as depicted in SEQ ID No. 1 or 2, or a functional variant thereof, and/or against an MRP14 polypeptide as depicted in SEQ ID No. 3 or 4, or a functional variant thereof.

While antibodies which can be used in accordance with the invention are known to the skilled person (e.g. Example 2, EP 0162 812; EP 585201; Deininger et al., 1999, J. Neuroimmunol., 93: 156-63), they can also be prepared using well-known methods: by immunizing a mammal, for example a rabbit, with the previously described MRP8 and MRP14 polypeptides or parts thereof having a length of at least 6 amino acids, preferably of at least 8 amino acids, in particular of at least 12 amino acids, where appropriate in the presence of e.g. Freund's adjuvant and/or aluminum hydroxide gels (see, e.g., Diamond, B. A. et al. (1981) The New England Journal of Medicine, 1344-1349). The polyclonal antibodies which are formed in an animal as a result of an immunological reaction can subsequently be readily isolated from the blood using well-known Do methods and be purified, e.g. by means of column chromatography. Monoclonal antibodies can be prepared, for example, using the known method of Winter & Milstein (Winter, G. & Milstein, C. (1991) Nature, 349, 293-299). According to the present invention, the term antibodies is also understood as meaning antibodies, or antigen-binding parts thereof, which are prepared recombinantly and modified, where appropriate, such as chimeric antibodies, humanized antibodies, multifunctional antibodies, bispecific or oligospecific antibodies, single-stranded antibodies and F(ab) fragments or F(ab)₂ fragments (see, e.g., EP-B1-0 368 684, U.S. Pat. No. 4,816,397, WO 88/01649, WO 93/06213, WO 98/24884). These antibodies can be used to investigate wound fluid readily and rapidly to determine whether one or both of the MRP8 and MRP14 polypeptides which can be used in accordance with the invention, is/are present in the wound fluid of an organism in a quantity which is reduced as compared with that in a normally healing wound in order, thereby, to obtain an indication of a possible wound-healing disturbance (see Example 2). For detecting the antibodies according to the invention, they are labeled, for example, with an enzyme, as already described above. This makes it possible to detect the specific antibody-peptide complex readily and just as rapidly by way of an enzymic color reaction (see Example 2).

The invention furthermore relates to the use of an MRP8/MRP14 heterodimer, or of its individual components in combination, of at least on nucleic acid encoding an MRP8/MRP14 heterodimer or its individual components in combination, or of a cell which is expressing the entire heterodimer or its individual components in combination, for identifying at least one pharmacologically active substance which exerts an influence on the activity of MRP8/MRP14 heterodimers and/or their individual components in combination. For example, this can be effected by means of a system for testing the influence of potential pharmacologically active substances on the activity of MRP8/MRP14 heterodimers, or their individual components in combination, in cells of the skin, in particular keratinocytes, monocytes, neutrophilic granulocytes, fibroblasts and endothelial cells.

In a preferred embodiment, at least one MRP8/MRP14 heterodimer which can be used in accordance with the invention, or its individual components in combination, is expressed by at least one cell and at least one substance is examined for its pharmacological activity.

For example, an investigation can be carried out to determine whether the calcium-binding activity of MRP8/MRP14 heterodimers, or their individual components in combination, is altered by applying substances. Thus, the cells can, for example, be labeled in vivo with radioactive calcium and the MRP8/MRP14 heterodimers, or their individual components in combination, can be isolated, before or after treating the cells with the substances, and examined by means of scintillation for the presence of radioactively bound calcium. Another functional assay can consist in analyzing the binding of keratin in the cell before and after applying the substances, as described in Goebeler et al. (Biochem J., 1995, 309: 419-24). Furthermore, it is possible, by means of double-staining untreated or treated cells with an anti-tubulin antibody and an anti-MRP8 antibody, an anti-MRP14 antibody or an anti-MRP8/MRP14 antibody to examine, using a suitable detection system, whether substances exert an influence on tubulin binding, which is essential for the polypeptides to be secreted. In addition, it is possible to test whether the quantity of extracellular, secreted MRP8/MRP14 heterodimers, or their individual components in combination, is altered by applying substances to the cells. In the case of murine MRP8, this polypeptide can, for example, be purified, or partially purified, from the cells, after these substances have been applied, and the isolate can be examined for chemotactic activity, with this activity being compared with that of control MRP8 which has been obtained from untreated cells (Lackmann et al., 1992, J. Biol. Chem., 267, 7499-7504).

Particular preference is to given to test systems which are suitable for identifying substances which increase the activity and/or secretion of MRP8 and/or MRP14 and which have as little influence as possible on the activity and/or the secretion of one or more control polypeptides such as GAPDH.

Another suitable test system which can be used in accordance with the invention is based on identifying interactions with the two hybrid system (Fields and Stemglanz, 1994, Trends in Genetics, 10, 286-292; Colas and Brent, 1998 TIBTECH, 16, 355-363). In this test system, cells are transformed with expression vectors which express fusion proteins which consist of at least one polypeptide according to the invention and a DNA-binding domain of a transcription factor such as Gal4 or LexA. The transformed cells also contain a reporter gene whose promoter contains binding sites for the corresponding DNA-binding domain. By means of transforming a further expression vector, which expresses a second fusion protein consisting of a known or unknown polypeptide and an activation domain, for example from Gal4 or herpes simplex virus VP16, the expression of the reporter gene can be greatly increased if the second fusion protein interacts with the investigated polypeptide according to the invention. This increase in expression can be used for identifying new interacting partners, for example by preparing a cDNA library from wound tissue for the purpose of constructing the second fusion protein. In a preferred embodiment, the interaction partner is an activator of MRP8/MRP14 This test system can also be used for screening substances which inhibit an interaction between the polypeptide according to the invention and an interacting partner. Such substances decrease the expression of the reporter gene in cells which are expressing fusion proteins of the polypeptide according to the invention and the interacting partner (Vidal and Endoh, 1999, Trends in Biotechnology, 17: 374-81). In this way, it is possible to rapidly identify novel active compounds which can be employed for the therapy of and/or prevention of skin diseases, wounds and/or wound-healing disturbances having a reduced quantity of MRP8/MRP14 heterodimers; perferably for the therapy of and/or prevention of diabetes-associated badly-healing wounds.

The invention also relates to a functional assay on the activity of human MRP8/MRP14 heterodimer or its individual components in combination, or of at least one nucleic acid encoding the heterodimer or its individual components in combination, comprising the steps of:

1) bringing the human MRP8/MRP14 heterodimer or its individual components in combination, or the at least one nucleic acid encoding the entire heterodimer or its individual components in combination into contact with at least one cell,

2) treating the at least one cell with at least one test substance,

3) measuring migration of the at least one cell, and

4) comparing the measured migration of the at least one cell treated with the at least one test substance with the migration of at least one control cell which was/were not treated with the at least one test substance.

In a preferred embodiment of the test system at least one human MRP8/MRP14 heterodimer which can be used in accordance with the invention, or its individual components in combination are brought into contact with the at least one cell.

In another preferred embodiment of the test system, at least one polynucleotide encoding a human MRP8/MRP14 heterodimer or its individual components in combination or parts thereof or variants thereof is brought into contact with the at least one cell. For example polypeptides useable according to the invention may be isolated from tissue or recombinantly produced and isolated by methods well known to the person skilled in the art. Following production, isolation and/or purification polypeptides according to the invention can be administered to the at least one cell. Methods for bringing an polynucleotide into a cell are well known to a skilled person and are described above. For example, expression vectors can be used to increase the expression a human MRP8/MRP14 heterodimer or its individual components in combination or parts thereof. Also it is possible, for example to use antisense nucleotides to decrease the expression of a human MRP8/MRP14 heterodimer or its individual components in combination. The modulation of expression can be transient, stable or inducible.

In a preferred embodiment of the test system, the cells are skin cells, especially keratinocytes, fibroblasts or endothelial cells. Preferably cell function is proliferation, migration or differentiation, especially migration. Especially preferred is a migration assay with keratinocytes using the MRP8/MRP14 heterodimer (see Example 4). Migration can be established readily by means of the “migration index” test (Charvat et al., see above) and comparable test systems (Benestad et al., 1987, Cell Tissue Kinet. 20: 109-19; Junger et al., 1993, J. Immunol. Methods 160: 73-9). Example for migration assays are well known to the skilled person and comprise for example the Boyden chamber method (Example 4), the scratch assay, the colloidal gold assay and an assay based on the migration in a fibrin matrix. In a scratch assay, cells are seeded on a tissue culture plate and are grown to confluency. The confluent cell layer is then wounded under standard conditions with a plastic pipet tip to create a cellfree zone. Subsequently, test substances can be added after and migration into the cellfree zone can be monitored by photo documentation of identical locations in the scratch. For the colloidal gold assay, coverslips are coated with colloidal gold salts and covered with a suitable substratum, for example Collagen I. Cells, for example keratinocytes are plated on the cover slip and allowed to migrate for several hours. Afterwards the cells are fixed in formaldehyde and migration tracks can be analysed using computer assisted image analysis. In the assay based on the migration in a fibrin matrix, cells are plated onto a fibrin matrix, that is obtained from freeze-dried surgical fibrinogen and distributed onto culture dishes before clotting. The fibrin matrix is transparent and therefore suitable for microscopic analysis of the cells. Suitable cells, for example keratinocytes are incubated on the matrix for 24 hours, fixed with formaldehyde and tunnels generated by migrating cells in the matrix are examined by light microscopy. Test systems for the monitoring of differentiation depend on the cell types and are well known for skilled persons for many cell types. For example, several test systems are available for keratinocytes: examples of suitable differentiation markers are keratin 6, 10 and 14 and also loricrin and involucrin (Rosenthal et al., 1992, J. Invest. Dermatol. 98: 343-50) whose expression can be readily detected, for example, using generally available antibodies. Suitable test systems for measuring proliferation can be established very rapidly by means, for example, of the incorporation of labeled nucleotides into the DNA of the cells (see, e.g., Savino and Dardenne, 1985, J. Immunol. Methods 85: 221-6; Perros and Weightman, 1991, Cell Prolif. 24: 517-23; de Fries and Mitsuhashi, 1995, J. Clin. Lab. Anal. 9: 89-95) by staining the cells with specific dyes (Schulz et al., 1994, J. Immunol. Methods 167: 1-13) or by means of immunological methods (Frahm et al., 1998, J. hnmunol. Methods 211: 43-50).

A migration assay on the function of MRP8/MRP14 heterodimer or its individual components in combination (see e.g. Example 4) can also be used for monitoring the activity of purified or recombinantly expressed MRP8/MRP14, e.g. in a quality assurance procedure. Quantitatively defined amounts of prepared MRP8/MRP14 heterodimer or its individual components in combination can be tested for functionality by bringing the human polypeptide(s) and/or the human nucleic acid(s) to be tested into contact with keratinocytes. Migration of the keratinocytes is quantified and then compared with the expected, standard migration value. Only preparations which exert the expected standardized effect on migration are allowed for manufacturing for example of therapeutic or diagnostic agents.

In a further preferred embodiment, at least one MRP8/MRP14 heterodimer which can be used in accordance with the invention, or its individual components in combination, or at least one cell expressing the entire heterodimer, or its individual components in combination is bound to a solid phase and at least one substance is examined for its pharmacological activity. The binding to a solid phase can be effected, for example, in the form of an array. Methods for preparing such arrays using solid phase chemistry and photolabile protecting groups has been disclosed, for example, in U.S. Pat. No. 5,744,305. Suitable assays for examining a pharmacological effect of test substances on an MRP8/MRP14 heterodimer which is bound to a solid phase, or its individual components in combination, are, for example, a fatty acid-binding assay for the MRP8/MRP14 heterodimer, known from Kerkhoff et al. (J. Biol. Chem., 1999, 274: 32672-32679), or a calcium-binding assay using ⁴⁵Ca²⁺.

It is furthermore possible to examine, in a wound-healing assay, for example carried out in mice, whether the application of MRP8/MRP14 heterodimers, or their individual components in combination, or of at least one nucleic acid encoding an MRP8/MRP14 heterodimer, or its individual components in combination, or of a cell which is expressing the entire heterodimer, or its individual components in combination, together, or at different time points, with test substances, to a wound alters the healing of the wound. This can be done, for example, by measuring the rate of reepithelialization, the amount of collagen deposition or the the determination of wound breaking strength.

The invention also relates to the use of at least one nucleic acid encoding an MRP8/MRP14 heterodimer, or its individual components in combination, or of a cell which is expressing the entire heterodimer or its individual components in combination, in combination, for identifying at least one pharmacologically active substance which exerts an influence on the expression of MRP8/MRP14 heterodimers, mRNAs encoding them, and/or their individual components in combination, or mRNAs encoding them.

Assays for identifying pharmacological substances which exert an influence on the expression of genes are well known to the skilled person (see, for example, Sivaraja et al., 2001, U.S. Pat. No. 6,183,956). Thus, cells which express MRP8 and/or MRP14, for example granulocytes, can be cultured as a test system for analyzing gene expression in vitro, with preference being given to skin cells, in particular keratinocytes, fibroblasts or endothelial cells. In this context, a possible test system is the human keratinocyte cell line HaCaT, which is available generally. Gene expression is analyzed, for example, at the level of the mRNA or of the polypeptides. In this connection, the quantity of MRP8 and/or MRP14 mRNA or polypeptide present after adding one or more substances to the cell culture is measured and compared with the corresponding quantity in a control culture. This is done, for example, with the aid of the hybridization of an antisense probe (see Example 1), which can be used to detect the MRP8 and/or MRP14 mRNA which is present in the lysate of the cells. The hybridization can be quantified, for example, by binding a specific antibody to the mRNA-probe complex (see Stuart and Frank, 1998, U.S. Pat. No. 4,732,847). In this connection, it is possible to carry out the analysis as a high-throughput method and to analyze a very large number of substances for their suitability as modulators of the expression of MRP8/MRP14 (Sivaraja et al., 2001, U.S. Pat. No. 6,183,956). In this connection, the substances to be analyzed can be taken from substance libraries (see, e.g. DE19816414, DE19619373) which can contain several thousand substances which are frequently very heterogeneous. Alternatively, the entire RNA or mRNA can first of all be isolated from cells and the absolute quantity, or the relative proportion, of the MRP8 and/or MRP14 mRNA can then be determined, for example by means of quantitative RT-PCR (see EP 0 200 362; Wittwer et al., 1997, BioTechniques 22: 130-8; Morrison et al., 1998, BioTechniques 24: 954-62) or by means of the RNAse protection assay (see, e.g., Sambrook et al., 1989, Molecular Cloning: A Laboratory Manual, Cold Spring Harbor, Cold Spring Harbor Laboratory Press, New York, chapter 7; EP 0 063 879). Another possibility is that of analyzing the quantity of polypeptide in the cell lysate using antibodies which specifically recognize MRP8 and/or MRP14 (see Example 2). In this case, quantification can be effected using, for example, an ELISA or a Western blot, which are well known. In order to determine the specificity of the substances for the expression of MRP8 and/or MRP14, the influence exerted by substances on the expression of MRP8/MRP14 can be compared with their influence on the expression of other genes, such as metabolism genes such as GAPDH. This can either be done in separate analyses or in parallel with the analysis of the MRP8/MRP14 heterodimer or its individual components in combination. Particular preference is to be given to assays which are suitable for identifying substances which increase the expression of MRP8 and/or MRP14 and which exert as little influence as possible on the expression of one or more control genes, such as GAPDH.

Another embodiment of the invention relates to the pharmacologically active substances which are identified with the aid of the screening methods.

The invention furthermore relates to a drug which comprises pharmacologically active substances for treating skin diseases, wounds and/or wound-healing disturbances having a reduced quantity of MRP8/MRP14 heterodimers, in particular diabetes-associated wounds.

In a preferred embodiment, at least one MRP8/MRP14 heterodimer which can be used in accordance with the invention, or its individual components in combination, is/are bound to a solid phase and at least one substance is examined for its pharmacological activity.

In another preferred embodiment, at least one MRP8/MRP14 heterodimer which can be used in accordance with the invention, or its individual components in combination, is/are expressed by at least one cell and at least one substance is examined for its pharmacological activity.

In a particularly preferred embodiment of the invention, at least two substances are, for the purpose of identifying pharmacological substances, examined for their pharmacological activity, with the substances being selected from at least one library of substances.

The invention furthermore relates to a process for producing a drug, with, in a first step, a pharmacologically active substance being identified using one of said methods for identifying such substances and, in a second step, the pharmacologically active substance which has been identified being brought into contact or combined with suitable auxiliary substances and/or additives.

The invention will now be further clarified with the aid of the table and examples which follow without it being restricted thereto.

### TABLE, FIGURES AND SEQUENCES

Table 1: shows the SEQ ID numbers and access numbers of the polypeptides which can be used in accordance with the invention and the cDNAs which encode them.

FIG. 1: Determination of MRP8/MRP14 polypeptide concentrations in the wound fluid of healthy patients, in the wound fluid of patients suffering from chronic diabetic wounds and in the wound fluid of patients suffering from venous ulcer, by means of sandwich ELISA.

FIG. 2: Results of the genetherapeutic treatment of diabetic rats with MRP8/MRP14. The plot depicts E/C values of the breaking strength, each value representing an E/C value of an individual treated wound compared to a control wound at day 7 after injury.

FIG. 3: Results of the genetherapeutic treatment of diabetic rats with MRP8/MRP14. The plot depicts E/C values of the breaking strength, each value representing an E/C value of an individual treated wound compared to a control wound at day 10 after injury. The mean value is depicted as a horizontal line.

SEQ ID No. 1 to SEQ ID No. 8 show the sequences of the murine and human MRP8 and MRP14 polypeptides and the cDNAs encoding them.

SEQ ID No. 9 to SEQ ID No. 18 show the sequences of the oligonucleotides which were used for the experiments.

### EXAMPLES

### Example 1

### Localization of MRP14 mRNA in Human Biopsies Taken from Healthy Skin and from a Wound in a Healthy Test Subject, and in Biopsies Taken from a Venous Ulcer and a Diabetic Ulcer patient

MRP14 is a component of the heterodimer MRP8/MRP14. An experiment was carried out to investigate the extent to which the expression of MRP14 is regulated differentially in various wound-healing diseases. For this, skin samples were taken, using 4 mm and 6 mm punches, from untreated intact skin and the day 5 wounds, respectively, of 4 healthy test subjects. In addition, punch biopsies of both intact skin and of the wound were taken from a patient with a venous ulcer and from a patient with a diabetic ulcer.

The localization of the MRP14 mRNA was investigated by means of nonradioactive in situ hybridization. In order to prepare the hybridization probe, a partial human MRP14 cDNA fragment was amplified by PCR. The primers which were used in this context contained an RNA polymerase promoter, for preparing riboprobes, in addition to the segment which was homologous to MRP14 (antisense primer: Sp6-MRP-14 primer (ATTTAGGTGACACTATAGAATAC CCC GAG GCC TGG CTT ATG GT; SEQ ID No. 9); control-sense primer: T3-MRP-14 primer (AATTAACCCTCACTAAAGGGG GTG GCT CCT CGG CTT TGA CA; SEQ ID No. 10). The amplified cDNA fragment was cloned into the vector pCR 2.1 TOPO (invitrogen) and the match of the insert with MRP14 was subsequently verified by sequencing. The antisense riboprobe and the sense control probe were prepared using the “DIG RNA labelling mix” (S. Hoffmann-La Roche) and the respective RNA polymerase in accordance with the manufacturer's instructions. The subsequent in situ hybridization was carried out as described in Komminoth et al. (1992, Histochemistry 98: 217-228).

The experiment showed that, while it was not possible to detect any expression of MRP14 in the intact skin of the healthy test subjects, significant labeling was observed in the normally healing wounds of these patients: the MRP14 mRNA was strongly expressed in the suprabasal cell layers of the hyperproliferative epithelium in biopsies from the healthy test subjects whereas no labeling was observed when using the sense control probe. Analysis of the ulcer biopsies showed that no labeling was detectable in the intact skin of the ulcer patients as well as in the intact skin of the healthy patients. Strong labeling was observed at the edge (corresponds to the hyperproliferative epithelium in normally healing wounds) of the venous ulcer wound. This demonstrates that there is no aberrant regulation in the venous ulcer. “Aberrant regulation” of the MRP8/MRP14 heterodimers, and/or their individual components in combination, in association with skin diseases, wounds and/or wound-healing disturbances is defined as a strength of expression which turns out to be markedly reduced, as compared with that seen in normal wound healing, in the cells, in the body fluids, in the wound liquid and/or in the skin. By contrast, a different result was surprisingly observed in the case of the labeling of the diabetic ulcer: only very weak labeling as observed at the edge of the wound. This shows that MRP14 is only expressed to a decreased extent at the edge of the diabetic ulcer wound but not at the edge of the venous ulcer wound or in normally healing wounds. The fact that the lack of MRP14 is observed in the cell layer which is required for proliferation and thus reepithelialization indicates that the wound-healing disturbance in diabetic patients could be caused by an inhibition of proliferation due to the content of MRP8/MRP14 polypeptide being too low.

It is therefore possible to successfully and effectively treat skin diseases, wounds and/or wound-healing disturbances having a reduced quantity of MRP8/MRP14 heterodimers, in particular diabetes-associated wounds, by compensating for the lack of MRP14 by adding MRP8/MRP14 heterodimer polypeptide or its components in combination or nucleic acids encoding these.

### Example 2

### Detecting MRP8 polypeptide in Wound Fluid from Various Wound-Healing Diseases

The aim now was to test the result obtained in Example 1 as far as the binding partner MRP8 was concerned. For this, wound fluid was isolated, by means of drainage, from normally healing human day 1 and day 2 wounds, as was wound fluid from a poorly healing wound of a diabetes patient and of a patient with a venous ulcer and the quantity of MRP8 in the wound fluids was determined by means of Western blot analysis. Biopsy tissue from a normally healing human day 5 wound, which was isolated as described in Example 1, was used as a positive control for detecting the polypeptide.

The wound fluid from normally healing wounds and from the wound in the diabetic patient was isolated from subcutaneous tissue by means of redon drainage after 1 day and after 2 days. The wound fluid from the patient with a chronic ulcer was obtained by vacuum-sealing the wound and drawing off the fluid through the vacuum after 1 day and after 2 days. A centrifugation was first of all carried out at 200×g (4° C.; 15 min) in order to remove relatively large cell constituents. The supernatant was decanted and centrifuged once again in order to remove the remaining cell debris (3300×g (4° C.; 15 min). The samples were then stored at −80° C. After thawing, the polypeptide concentrations in the samples were determined by means of a BCA test (Sigma-Aldrich Chemicals) and the same quantities of polypeptide were loaded onto a 4-20% gradient gel (tris-glycine buffered, 1 mm, Novex) and the polypeptides were fractionated electrophoretically. After blotting, an immunostaining was carried out using the polyclonal goat anti-human calgranulin A antibody (dilution 1:100; Santa Cruz, sc8112), as the first antibody, and the donkey anti-goat IgG F(ab′) fragment (dilution 1:5000; Dianova, # 705-036-147) as the second anti-body. The signal was detected using the “Amersham ECL Western Blotting detection reagent” (Amersham, # RPN 2166) in accordance with the manufacturer's instructions.

Both in the case of the sample which was obtained from the biopsy tissue from the normally healing wound and in the case of the samples which contained the wound fluid from normally healing day 1 and day 2 wounds, a strong signal was observed at approx. 9 kDa. A signal intensity which was increased by a factor of 2 was detected in the day 2 wound fluid as compared with the day 1 wound fluid. This thereby demonstrated, for the first time, that MRP8 is present in wound fluid. MRP8 is secreted into the extracellular medium by epidermal cells. As was already demonstrated in Example 1 as far as MRP14 was concerned, examination of the wound fluid obtained from the venous ulcer showed that there was no difference in expression strength between the venous ulcer and a normally healing wound. This once again confirms that there is no lack of MRP8/MRP14 in venous ulcers. By contrast, it was possible to demonstrate that there was a lack of MRP8 polypeptide in the wound fluid obtained from the poorly healing wound of the diabetic patient. It was scarcely possible to detect any signal in this sample following immunostaining. Taken together with the result from Example 1, this proves that there is aberrant regulation of the MRP8 and MRP14 polypeptides at the mRNA and polypeptide levels, and consequently also a lack of MRP8/MRP14 heterodimers, in diabetic wounds.

On the other hand, this experiment demonstrates that, in wounds, MRP8/MRP14 is secreted in quantity into the extracellular medium, i.e. the wound fluid. Taken together with the finding that there is a lack of MRP8 in the wound fluid of diabetic wounds, it follows from these results that the quantity of MRP8/MRP14 mRNA and/or polypeptide in the wounds of diabetic patients, preferably the quantity of polypeptide in the wound fluid, has to be raised in order to effectively treat skin diseases, wounds and/or wound-healing disturbances having a reduced quantity of MRP8/MRP14 heterodimers, in particular diabetes-associated wounds, or to prevent the development of a poorly healing wound or indeed of a diabetic ulcer.

### Example 3

### Gene-Therapeutic Treatment of Normally Healing and Diabetic wounds in Rabbits with MRP8/MRP14

In order to confirm that MRP8/MRP14 is in fact particularly suitable for treating and/or preventing skin diseases, wounds and/or wound-healing disturbances having a reduced quantity of MRP8/MRP14 heterodimers, in particular diabetes-associated wounds which heal poorly, the influence of MRP8/MRP14 on wound healing in vivo was investigated in untreated and diabetic male New Zealand White rabbits. The alloxan administration-induced diabetic “New Zealand White rabbit model system” is suitable for simulating diabetes-associated wound-healing disturbances since it is a well investigated, scientifically established model system in which the state of health of the animals is comparable and wound closure is significantly retarded, namely by 50% (Davidson, 1998, Arch. Derm. Res., 290: S1-S11). The diabetic state is induced by administering alloxan which results in the selective destruction of the Langerhans cells and consequently leads to irreversible hyperglycemia. Diabetes is induced after starving the animals for 12 h. For this, alloxan (70-75 mg/kg of body weight) was administered intravenously as an aqueous solution. After the injection, the animals were given a 10% glucose solution for drinking, together with a block of sugar, for 48 hours and food and water were made available ad libitum. The blood sugar level was determined (Glukosemeter, Bayer AG) 4 and 7 days (wounding day) after the alloxan injection. A value of more than 200 mg/dL was judged to be indicative of the diabetic state. Wounding and gene-therapeutic treatment then followed.

In one experiment, an expression vector, pMRP8/MRP14 (see below), which contained the two murine cDNAs (SEQ ID No. 5 and SEQ ID No. 7), was used for this purpose. In another experiment, an MRP8-containing vector, pMHintMRP8 (see below), was shot, together with an MRP14-containing vector, pMHintMRP14 (see below), into a wound using a gene gun. The wound healing was investigated on the basis of the rate at which the wound reepithelialized, which rate was determined by means of EPICAM photographs. In order to obtain pMHintMRP8 and pMHintMRP14, a suitable expression vector, pMHint, was first of all constructed, with this vector being prepared from vector pMH (S. Hoffmann-La Roche) by inserting intron II of the rat insulin gene into the HindIII cleavage site between the CMV promoter and the multiple cloning site. The murine MRP8 or MRP14 cDNA was then cloned into pMHint using the multiple cloning site. For this, the MRP8-encoding region of the MRP8 cDNA was amplified by PCR (MRP8 primer 1: GAG AGA GGT ACC ATG CCG TCT GAA CTG GAG (SEQ ID No. 11) and MRP8 primer 2: GAG AGA GAC ACG TGC TAC TCC TTG TGG CTG TCT TTG (SEQ ID No. 12)), then cut with KpnI and PmlI and ligated to the expression vector pMHint, which had been cut with KpnI and PmlI, thereby giving rise to the expression plasmid pMHintMRP8. In order to obtain pMHintMRP14, the coding region was likewise amplified by PCR (MRP14-primer 1: GAG AGA GGT ACC ATG GCC AAC AAA GCA (SEQ ID No. 13) and MRP14-primer 2: GAG ACC CGG GTT ACT TCC CAC AGC CTT TG (SEQ ID No. 14)). The resulting product was cut with KpnI and SmaI and ligated to the expression vector pMHint, which had been cut with KpnI and PmlI, thereby giving rise to the expression plasmid pMHintMRP14.

In order to obtain the plasmid pMRP8/MRP14, MRP14 was first of all cloned into the vector pBudCE4.1 (Invitrogen), which contains both a CVM promoter and an EF-1 alpha promoter for expressing two polypeptides in eukaryotic cells. The coding sequence of MRP14 was amplified by PCR (MRP14-primer 3: GAGAGAGGTA CCATGGCCAA CAAAGCA (SEQ ID No. 15); MRP14-primer 4: GAGAGACTCG AGTTACTTCC CACAGCCTTT G (SEQ ID No. 16)), cut with KpnI and XhoI and ligated to the vector, which had been cut with KpnI and XhoI. The coding sequence of MRP8 was subsequently amplified by PCR (MRP8-primer 3: GAGAGAGTCG ACATGCCGTC TGAACTGGAG (SEQ ID No. 17); MRP8-primer 4: GAGAGAAGTA CTCTACTCCT TGTGGCTGTC TTTG (SEQ ID No. 18)), cut with SalI and ScaI and ligated to the vector, which had been cut with SalI and ScaI and already contained MRP14 cDNA.

Alloxan-treated rabbits were used for investigating the influence of MRP8, together with MRP14, on wound healing in diabetic animals. In this experiment, the MRP8 and MRP14 nucleic acids which would be expressed in the wound were applied to the wound either located on separated vectors or located on a common vector.

Diabetic animals, and also the untreated animals, were anesthetized with xylazine and ketamine (4-5 and 50-70 mg/kg i.m., respectively) (subsequent dosage was with ketamine) and treated with depilation cream. Adrenaline solution (2% xylocain solution and epinephrine, 1:100,000) was subsequently injected intradermally in order to constrict the blood vessels and to separate the skin from the ear cartilage lying below it. Punches were then used to make four 8 mm wounds on the inside of the ear. Each wound was treated at a pressure of 500 psi using a Helios Gene Gun (BioRad), with 0.5 μg of control plasmid or expression plasmid, which had been immobilized on gold particles (BioRad) being used per shot. 0.5 μg of MRP8/MRP14 were used per shot in the case of the experiment using the vector pMRP8/MRP14; in the case of the experiment using pMHintMRP8 and pMHintMRP14, 0.25 μg of expression plasmid was in each case immobilized on gold particles and shot into the wound together. The wounds were subsequently covered with a semiocclusive dressing. On day 10 after wounding, the wound surface which had still not completely reepithelialized was determined by means of EPICAM and compared with the value at day 0 (immediately after wounding).

In the case of the normally healing rabbits, the experiment in which the vector MRP8/MRP14, expressing MRP8 and MRP14, has been used, gave a reepithelialization rate (mm²/day) which was improved by 15% as compared with that of the control. Surprisingly, after having been bombarded with the plasmid, the diabetic animals, which normally exhibit a greatly reduced rate of wound healing, achieved a rate of reepithelialization which was precisely as high as that achieved in the normally healing rabbits whose wounds were treated with vector pMRP8/MRP14 expressing MRP8 and MRP14. This means that the wound-healing disturbance in these animals was not only compensated for by the treatment with MRP8/MRP14 but that this treatment even resulted in the achievement of the same markedly improved rate of wound healing which was achieved after treating the normally healing animals with MRP8/MRP14. This clearly proves the special efficacy and suitability of MRP8 and MRP14 especially in combination for treating and/or preventing skin diseases, wounds and/or wound-healing disturbances having a reduced quantity of MRP8/MRP14 heterodimers, in particular diabetes-associated wounds. It was also possible to confirm this result in the second experiment in which the wounds were treated simultaneously with both the MRP8-expressing vector pMHintMRP8 and the MRP14-expressing vector pMHintMRP14 whereby MRP8 and MRP14 were administered separately on two different vectors.

Normally, only a few cells are transfected in association with a gene therapy treatment. Despite this, it was possible to demonstrate greatly improved wound healing precisely in the poorly healing wounds of the diabetic animals. This shows that the MRP8/MRP14 dimer, which is secreted and thereby distributed into the wound fluid, is effective and that the effect of the administration of the vector is not restricted to the few cells which took up the vector and expressed MRP8/MRP14. This experiment furthermore proves that the extracellular deficiency of the MRP8/MRP14 in mammals has to be preferentially redressed in order to effectively treat skin diseases, wounds and/or wound-healing disturbances having a reduced quantity of MRP8/MRP14 heterodimers, in particular diabetes-associated wounds.

It was, in fact, possible to demonstrate that treating the wounds of both normal rabbits and diabetic rabbits with pMHintMRP8 together with pMHintMRP14 resulted in identical reepithelialization rates which turned out to be 17% higher than the reepithelialization rate seen in normally healing, untreated rabbits. In turn, therefore, it was possible to observe a significant improvement in the wound healing of the diabetic animals, with this improvement going beyond the rate of wound healing which is to be expected in healthy animals. At the same time, this result proves that skin diseases, wounds and/or wound-healing disturbances having a reduced quantity of MRP8/MRP14 heterodimers, in particular diabetes-associated wounds, can be treated very effectively by increasing the quantity of MRP8/MRP14 heterodimers.

### Example 4

### In Vitro assay for Human MRP8/MRP14 Suitable for the Identification of Pharmacologically Active Substances

The extracellular function of human MRP8 and MRP14 in combination is unknown. Therefore, it is also object of the present invention to identify an assay, which allows the identification of pharmacologically active substances, which modulate the extracellular function of human MRP8 and MRP14 in combination. In particular, such an assay may be used to identify substances which increase the activity of human MRP8 and MRP14 in combination, as such substances may be used for the treatment of diseases characterized by reduced levels of MRP8 and/or MRP14, especially diabetic wounds. Substances, which modulate the extracellular function of human MRP8 and MRP14 in combination have the advantage that they need not enter the cell for exerting their influence, but may be applied easily topically.

Surprisingly, it was found that MRP8 in combination with MRP14 have a positive effect on the migration of keratinocytes, which was previously unknown and not suggested in the prior art, although the polypeptides are known for a long time and although they have been investigated in many respects. Therefore, the problem was solved by a migration assay using human MRP8 and MRP14 in combination as active substances and skin cells, especially keratinocytes, in particular HaCat cells. The migration assay may also be used to test whether variants of human MRP8 and MRP14 in combination are functional variants according to the present invention.

As an example, a migration assay using the Boyden chamber method was used. It is the principle of the Boyden chamber migration assay to measure a cell movement towards a polypeptide gradient. The classical Boyden chamber consists of two compartments, which are separated by a filter with defined pore size. The lower compartments is filled with a chemoattractant, the upper compartments with cell suspension. During incubation the cells of the upper chamber migrate through the filter towards the polypeptide gradient. At the end of the assay the cells on the lower side of the filter are quantified. The assay employing this principle used here is a Transwell Assay (Chemicon International, Inc.) which consists of chamber inserts with filters in a 24 well tissue culture dish. The filters obtained by the manufacturer are coated with bovine Collagen I and have a pore size of 8 μm. The putative chemoattractant used was MRP8 in combination with MRP14 in different concentrations and cells used were HaCaT keratinocytes. HaCaT keratinocytes cells were cultured in Dulbeccos modified Eagles Medium (DMEM, Gibco) with 10% foetal calf serum (FCS, Gibco). The cells were splitted every 7 days in a 1:10 ratio and medium was changed every second day. 24 hours prior to the assay, cells were trypsinized and 5 million cells were seeded in a 75 cm² flask. This procedure facilitates cell detachment and impairs the formation of cell aggregates. To start the assay, cells were first treated with 0,05% EDTA (PAN) for 20 minutes and were then detached by treatment with trypsin (Gibco) for 2 minutes. After washing the cells to remove FCS, they were resuspended in DMEM without FCS but supplemented with 0.1% BSA (Merck) to prevent aggregation during the assay. The lower well, i.e. the lower compartment, of the Transwell chambers were filled with the chemoattractant (human MRP8 in combination with MRP14: non-phosphorylalted complex (isolated from human blood), phosphorylated complex (isolated from human blood), recombinant complex (expression in E. coli); concentration range:500 ng/ml, 5 μg/ml)). BSA (5 μg/ml) was used as negative control, EGF (Epidermal Growth Factor, R&D; 2 ng/ml) as positive control (Nickoloff et al., 1988, Am. J. Pathol., 132:543-551).

The upper compartment was filled with the HaCaT cell suspension: 2.5×10⁶ cells/well and incubated at 37° C. and 10% CO₂ for 4 hours. After incubation the filters were stained following the manufacturers instructions: Briefly, medium was removed from the compartment and cells on the upper side of the filter were carefully removed without puncturing the filter. The filters were then placed in the staining solution (Transwell Kit, Chemicon International, Inc.) for 30 minutes to stain the cells on the lower side of the filter which have migrated through the filter. Afterwards the filters were washed carefully with distilled water to remove residual staining solution from the filter. The staining solution absorbed by the cells was then extracted with extraction buffer (Transwell Kit, Chemicon International Inc.) by slowly shaking the filters on a shaker for 10 minutes. Finally, the absorbance of 100 μl of the extracted solution was measured in an ELISA reader at 570 nm. The intensity of the absorbance correlates with the number of cells migrated through the filter. In the case of human MRP8 in combination with MRP14, an induction of migration was detected for all three complexes, the highest induction being observable at a protein complex concentration of 5 μg/ml. The addition of the heterodimer resulted in a two-fold increase in the number of migrating cells compared to the negative control (BSA). Hence this Example shows, that the migration assay can be used for testing whether MRP8 or MRP14 variants in combination are functional variants according to the invention. For example, several migration assays may be performed in parallel.

### Example 5

### Determination of MRP8/MRP14 Concentrations by Sandwich ELISA in Wound Fluids

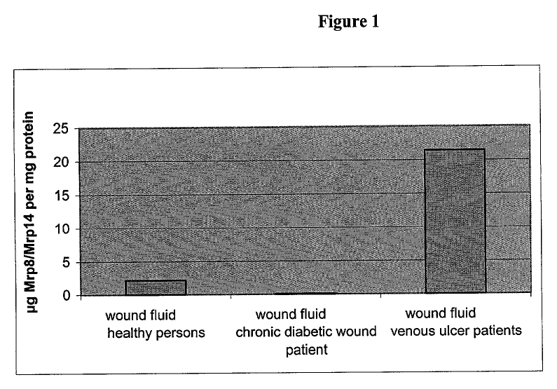

In order to determine the amount of MRP8/MRP14 polypeptide in various wound fluids, wound fluid samples from patients suffering from chronic diabetic wound were collected, venous ulcers as well as wound fluids from wounds of healthy persons. Wound fluids were isolated as described in Example 2 by means of drainage. For the determination of MRP8/MRP14 content, Maxi-Sorb Immunoplates (96-well, Nunc, Wiesbaden, Germany) were coated with polyclonal rabbit antisera against MRP14 by incubating each well overnight at 4° C. with 50 μl of coating solution (anti-MRP14 antibody, 2 μg/ml, in 0.1 M NaHCO3, pH 9.5). After extensive washing (3×) with phosphate-buffered saline (PBS), nonspecific binding of polypeptides was blocked with a solution of 0.25% bovine serum albumin (BSA, Sigma) and 0.1% Tween 20 (Sigma) in PBS for 1 h at room temperature. After removal of the blocking reagent, 50 μl of diluted wound fluid samples (buffer A: PBS containing 0.1% Tween 20) were added and allowed to react with the immobilized capture antibody for 1 h at 37° C. and then washed (3×) with buffer A. Bound antigen was detected by incubation (1 h, 37° C.) of each well with 50 μl of a biotinylated monoclonal antibody against MRP14 (clone S36.48, Dianova, 125 ng/ml antibody in buffer A). After another washing procedure (3×buffer A) the biotinylated antibody was detected by adding 50 μl peroxidase-conjugated Streptavidin (Jackson ImmunoResearch; 1/25,000 in buffer A for 30 min (1 h, 37° C.). The plates were extensively washed again (4×) before developing the colour reaction with 200 μl horseradish peroxidase reagent (25 ml citrate buffer, pH 4.0, 10 mg ABTS (2.2′ az-ino-di-(3-ethyl-benzthiazoline-6-sulfonate)) and 10 μl H₂O₂). Light absorption was measured at 405 nm with a microplate reader MRX (Dynatech, Denkendorf, Germany). For calibration, different amounts (0.25-250 ng/ml) of the native complex of human MRP8 and MRP14 were solubilized in dilution buffer and applied to the system. MRP8 and MRP14 spontaneously form noncovalently associated complexes to a quantitative extent which are detected by the ELISA system. Therefore, the ELISA is calibrated with the native MRP8/MRP14 complex and data are presented as microgram of MRP8/MRP14 per mg total polypeptide. The total polypeptide content per probe was determined using the BCA assay. The results are shown in FIG. 1.

Surprisingly it was found, that MRP8/MRP14 was almost absent in the wound fluid of the patient with the chronic diabetic wound, whereas the concentrations are clearly higher in wound fluids from normally healing wounds and even more elevated in wound fluids from venous ulcer patients.

### Example 6

### Genetherqpeutic Treatment of Diabetic Rats with MRP8/MPR14

In order to prove the suitability of the MRP8/MRP14 heterodimer for the treatment of diabetes-associated wounds, male diabetic Sprague Dawley rats (250-300 g) were treated with adenoviral constructs expressing murine MRP8 and MRP14, respectively (SEQ ID No. 5 and SEQ ID No. 7). The constructs were obtained using the AdEasy™ Technology (QBiogene) according to the manufacturer's protocol. In short, the cDNAs are amplified by PCR using primers which introduce KpnI and EcoRV sites at the end. Then, the PCR products are cut with KpnI and EcoRV and are subsequently cloned into the Transfer vector resulting in the vectors pShuttle-mrp8 and pShuttle-mrp14. Subsequently, recombinant adenoviruses were created according to the protocol, resulting in the constructs Ad-mrp8 and Ad-mrp 14. Expression of the polypeptides was verified by Western blot analysis of transfected QBI-293 cell lysate as described in the protocol. The adenoviral constructs were suspended and dialyzed in in HBS-buffer (20 mM HEPES/150 mM NaCl, pH 7,8).

For the in vivo experiments, rats were anaesthetized with an inhalant mixture consisting of 2% O₂ (2 L/min)+1.25% Isoflurane. After shaving and marking the wound sites on the back, incisional wounds with 1 cm in length were made. A mixture of adenoviral constructs carrying MRP8 (5*10⁸ PFU in 50 μl HBS) and MRP14 (5*10⁸ PFU in 50 μl HBS) cDNA was injected intracutaneously into the skin along the margin of incision. Each rat was treated with duplicate MRP8+MRP14 genes and the control adenoviral construct expressing lacZ (1*10⁹ PFU). Thus, each rat received a treatment with MRP8/MRP14 in an anterior and a posterior site as well as the LacZ control construct at an anterior or posterior site. The wounds were closed with wound clips and analyzed 7 or 10 days later for wound breaking strength using a BTC tensiometer. For each treated wound, the so-called E/C ratio of the breaking strengths of the wound treated with MRP8 and MRP14 and the corresponding wound treated with control construct was determined and the mean value of all E/C ratios was calculated. An E/C value>1 reflects a higher breaking strength of the treated wounds RELATIVE TO CONTROL WOUNDS and therefore indicates improved wound healing.

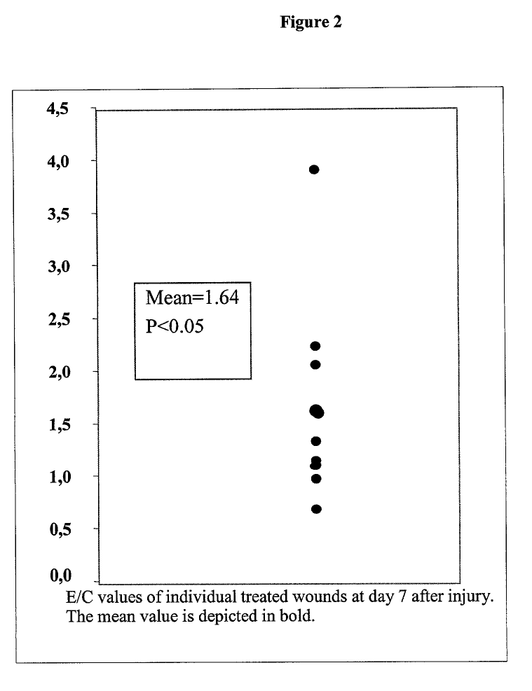

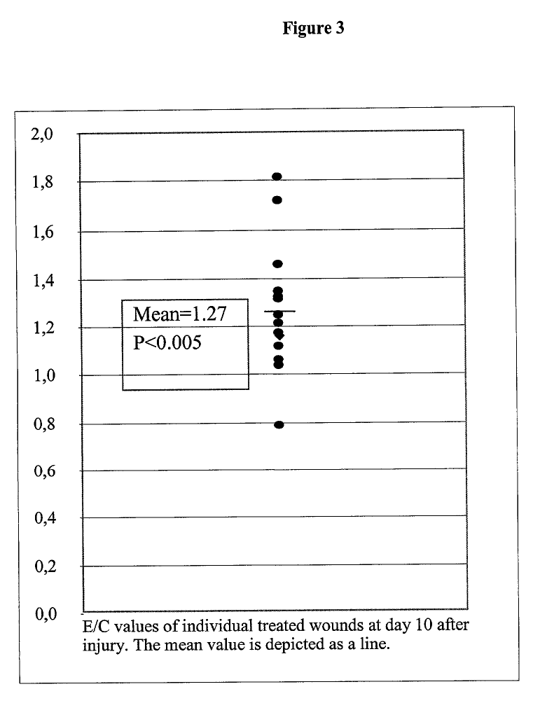

Surprisingly, it was found that the addition of MRP8 in combination with MRP14 leads to a strong and significant increase in breaking strength in diabetic rats 7 days as well as 10 days after injury and therefore, the heterodimer is especially suitable for treating diabetes-associated wounds by improving the mechanical properties of the wound (FIGS. 2 and 3): after 7 days, the mean E/C value was 1,64 (N=9 wounds; P<0,05) and after 10 days the E/C value was calculated 1,27 (N=14 wounds; P<0,005).

It will be apparent to those skilled in the art that various modifications can be made to the compositions and processes of this invention. Thus, it is intended that the present invention cover such modifications and variations, provided they come within the scope of the appended claims and their equivalents.

Priority application DE 10121254.2, was filed Apr. 30, 2001, and priority application U.S. Pat. No. 60/322925 was filed Sep. 17, 2001. All publications cited herein are incorporated in their entireties by reference.

TABLE 1

| | | | SEQ | | SEQ |
|---|---|---|---|---|---|
| | | | ID | | ID |
| Name | Organism | PROTEIN | No. | cDNA | No. |
| Murine | Mus muscu- | SwissProt: | 1 | EMBL: | 5 |
| MRP8/CP-10 | lus | P27005 | | M83218 | |
| Murine MRP14 | Mus muscu- | SwissProt: | 3 | EMBL: | 7 |
| | lus | P31725 | | M83219 | |
| Human MRP8 | Homo | SwissProt: | 2 | EMBL: | 6 |
| | sapiens | P05109 | | Y00278 | |
| Human MRP14 | Homo | SwissProt: | 4 | EMBL: | 8 |
| | sapiens | P06702 | | X06233 | |

1. A method of diagnosing a disease selected from the group consisting of skin diseases, wounds, and wound-healing disturbances, having a reduced quantity of MRP8/MRP14 heterodimers, in a patient, the method comprising determining in a sample from the patient, a level of at least one compound selected from the group consisting of the entire MRP8/MRP14 heterodimer, the individual components of the MRP8/MRP14 heterodimer in combination, at least one nucleic acid encoding the entire MRP8/MRP14 heterodimer, and at least one nucleic acid encoding the individual components of the MRP8/MRP14 heterodimer in combination, the level of the compound in the sample from the patient that differs from the level in a control sample indicating that said patient has the disease.

2. The method of diagnosing according to claim 1, wherein the wounds are diabetes-associated wounds.

3. The method of diagnosing according to claim 2, wherein the diabetes-associated wounds are a diabetic ulcer.

4. The method of diagnosing according to claim 1, wherein the MRP8/MRP14 heterodimer is employed in the form of a fusion protein.

5. The method of diagnosing according to claim 1, wherein the cell is an autologous or an allogenic cell.

6. The method of diagnosing according to claim 5, wherein the cell is a skin cell.

7. The method of diagnosing according to claim 6, wherein the skin cell is a cell selected from the group consisting of a keratinocyte, a fibroblast, and an endothelial cell.

8. A method of treating a disease selected from the group consisting of skin diseases, wounds, and wound-healing disturbances, having a reduced quantity of MRP8/MRP14 heterodimers, in a patient in need of the treatment, the method comprising administering to the patient at least one biomaterial selected from the group consisting of the entire MRP8/MRP14 heterodimer, the individual components of the MRP8/MRP14 heterodimer in combination, a nucleic acid encoding the entire MRP8/MRP14 heterodimer, a nucleic acid encoding the individual components of the MRP8/MRP14 heterodimer in combination, a cell expressing the entire MRP8/MRP14 heterodimer, and a cell expressing the individual components of the MRP8/MRP14 heterodimer in combination, in a therapeutically effective amount.

9. The method of treating according to claim 8, wherein the wounds are diabetes-associated wounds.

10. The method of treating according to claim 9, wherein the diabetes-associated wounds are a diabetic ulcer.

11. The method of treating according to claim 8, wherein the MRP8/MRP14 heterodimer is employed in the form of a fusion protein.

12. The method of treating according to claim 8, wherein the nucleic acid is employed in the form of an expression vector.

13. The method of treating according to claim 12, wherein the expression vector is a vector applicable in gene therapy.

14. The method of treating according to claim 8, wherein the cell is an autologous or an allogenic cell.

15. The method of treating according to claim 14, wherein the cell is a skin cell.

16. The method of treating according to claim 15, wherein the skin cell is a cell selected from the group consisting of a keratinocyte, a fibroblast, and an endothelial cell.

17. A method of identifying at least one pharmacologically active substance which exerts an influence on the function of an MRP8/MRP14 heterodimer or the individual components of the MRP8/MRP14 heterodimer in combination, the method comprising contacting at least one biomaterial selected from the group consisting of the entire MRP8/MRP14 heterodimer, the individual components of the MRP8/MRP14 heterodimer in combination, a nucleic acid encoding the entire MRP8/MRP14 heterodimer, a nucleic acid encoding the individual components of the MRP8/MRP14 heterodimer in combination, a cell expressing the entire MRP8/MRP14 heterodimer, and a cell expressing the individual components of the MRP8/MRP14 heterodimer in combination, with at least one substance and determining whether the substance exerts an influence on the function of the MRP8/MRP14 heterodimer or the individual components of the MRP8/MRP14 heterodimer in combination, identifying the at least one substance as the at least one pharmacologically active substance.

18. The method of identifying according to claim 17, wherein at least one biomaterial selected from the group consisting of the entire MRP8/MRP14 heterodimer, the individual components of the MRP8/MRP14 heterodimer in combination, a cell expressing the entire MRP8/MRP14 heterodimer, and a cell expressing the individual components of the MRP8/MRP14 heterodimer in combination, is bound to a solid phase and at least one substance is examined for its pharmacological activity.

19. The method of identifying according to claim 17, wherein the MRP8/MRP14 heterodimer, is expressed by at least one cell and at least one substance is examined for its pharmacological activity.

20. A method of identifying at least one pharmacologically active substance which exerts an influence on the expression of an MRP8/MRP14 heterodimer or the individual components of the MRP8/MRP14 heterodimer in combination, the method comprising contacting of at least one biomaterial selected from the group consisting of the entire MRP8/MRP14 heterodimer, the individual components of the MRP8/RP14 heterodimer in combination, a nucleic acid encoding the entire MRP8/MRP14 heterodimer, a nucleic acid encoding the individual components of the MRP8/MRP14 heterodimer in combination, a cell expressing the entire MRP8/MRP14 heterodimer, and a cell expressing the individual components of the MRP8/MRP14 heterodimer in combination, with at least one substance and determining whether the substance exerts an influence on the expression of the MRP8/MRP14 heterodimer or the individual components of the MRP8/MRP14 heterodimer in combination, identifying the at least one substance as the at least one pharmacologically active substance.

21. The method of identifying according to claim 20, wherein at least one biomaterial selected from the group consisting of an entire MRP8/MRP14 heterodimer, the individual components of the MRP8/MRP14 heterodimer in combination, a cell expressing the entire MRP8/MRP14 heterodimer, and a cell expressing the individual components of the MRP8/MRP14 heterodimer in combination, is bound to a solid phase and at least one substance is examined for its pharmacological activity.

22. The method of identifying according to claim 20, wherein at least one MRP8/MRP14 heterodimer, is expressed by at least one cell and at least one substance is examined for its pharmacological activity.

23. A functional assay on the activity of at least one component selected from the group consisting of a human MRP8/MRP14 heterodimer, the individual components of the MRP8/MRP14 heterodimer in combination, a nucleic acid encoding the MRP8/MRP14 heterodimer, and a nucleic acid encoding individual components of the MRP8/MRP14 heterodimer in combination, comprising the steps of:
1) bringing the at least one component into contact with at least one cell,
2) treating the at least one cell with at least one test substance,
3) measuring migration of the at least one cell, and
4) comparing the measured migration of the the at least one cell treated with the the at least one test substance with the migration of the at least one control cell which was not treated with the the at least one test substance.

24. A method of using the functional assay according to claim 23 for identifying the at least one pharmacologically active substance which exerts an influence on the activity of MRP8/MRP14 heterodimers or the individual components of the MRP8/MRP14 heterodimer in combination.

25. A method of using the functional assay according to claim 23 for identifying the at least one pharmacologically active substance which exerts an influence on the expression of MRP8/MRP14 heterodimers or the individual components of the MRP8/MRP14 heterodiner in combination.


SEQUENCE LISTING (18 sequences)

SEQ ID NO: 1 - protein, 89 aa, Mus musculus
MPSELEKALSNLIDVYHNYSNIQGNHHALYKNDFKKMVTTECPQFVQNINIENLFRELDINSDNAINFEEFLAMVIKVGVASHKDSHKE

SEQ ID NO: 2 - protein, 93 aa, Homo sapien
MLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 3 - protein, 113 aa, Mus musculus
MANKAPSQMERSITTIIDTFHQYSRKEGHPDTLSKKEFRQMVEAQLATFMKKEKRNEALINDIMEDLDTNQDNQLSFEECMMLMAKLIFACHEKLHENNPRGHGHSHGKGCGK

SEQ ID NO: 4 - protein, 114 aa, Homo sapien
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 5 - DNA, 270 bp, Mus musculus
atgccgtctgaactggagaaggccttgagcaacctcattgatgtctaccacaattattccaatatacaaggaaatcaccatgccctctacaagaatgacttcaagaaaatggtcactactgagtgtcctcagtttgtgcagaatataaatatcgaaaacttgttcagagaattggacatcaatagtgacaatgcaattaacttcgaggagttccttgcgatggtgataaaagtgggtgtggcatctcacaaagacagccacaaggagtag

SEQ ID NO: 6 - DNA, 285 bp, Homo sapien
atgttgaccgagctggagaaagccttgaactctatcatcgacgtctaccacaagtactccctgataaaggggaatttccatgccgtctacagggatgacctgaagaaattgctagagaccgagtgtcctcagtatatcaggaaaaagggtgcagacgtctggttcaaagagttggatatcaacactgatggtgcagttaacttccaggagttcctcattctggtgataaagatgggctggcagcccacaaaaaaagccatgaagaaagccacaaagagtagctga

SEQ ID NO: 7 - DNA, 342 bp, Mus musculus
atggccaacaaagcaccttctcagatggagcgcagcataaccaccatcatcgacaccttccatcaatactctaggaaggaaggacaccctgacaccctgagcaagaaggaattcagacaaatggtggaagcacagttggcaacctttatgaagaaagagaagagaaatgaagccctcataaatgacatcatggaggacctggacacaaaccaggacaatcagctgagctttgaggagtgtatgatgctgatggcaaagttgatctttgcctgtcatgagaagctgcatgagaacaacccacgtgggcatggccacagtcatggcaaaggctgtgggaagtaa

SEQ ID NO: 8 - DNA, 345 bp, Homo sapien
atgacttgcaaaatgtcgcagctggaacgcaacatagagaccatcatcaacaccttccaccaatactctgtgaagctggggcacccagacaccctgaaccagggggaattcaaagagctggtgcgaaaagatctgcaaaattttctcaagaaggagaataagaatgaaaaggtcatagaacacatcatggaggacctggacacaaatgcagacaagcagctgagcttcgaggagttcatcatgctgatggcgaggctaacctgggcctcccacgagaagatgcacgagggtgacgagggccctggccaccaccataagccaggcctcggggagggcaccccctaa

SEQ ID NO: 9 - DNA, 43 bp, Artificial Sequence
  Sp6-MRP-14 primer
atttaggtgacactatagaataccccgaggcctggcttatggt

SEQ ID NO: 10 - DNA, 41 bp, Artificial Sequence
  T3-MRP-14 primer
aattaaccctcactaaagggggtggctcctcggctttgaca

SEQ ID NO: 11 - DNA, 30 bp, Artificial Sequence
  MRP8 primer
gagagaggtaccatgccgtctgaactggag

SEQ ID NO: 12 - DNA, 36 bp, Artificial Sequence
  MRP8 primer
gagagagacacgtgctactccttgtggctgtctttg

SEQ ID NO: 13 - DNA, 27 bp, Artificial Sequence
  MRP 14 primer
gagagaggtaccatggccaacaaagca

SEQ ID NO: 14 - DNA, 29 bp, Artificial Sequence
  MRP14 primer
gagacccgggttacttcccacagcctttg

SEQ ID NO: 15 - DNA, 27 bp, Artificial Sequence
  MRP14 primer
gagagaggtaccatggccaacaaagca

SEQ ID NO: 16 - DNA, 31 bp, Artificial Sequence
  MRP14 primer
gagagactcgagttacttcccacagcctttg

SEQ ID NO: 17 - DNA, 30 bp, Artificial Sequence
  MRP8 primer
gagagagtcgacatgccgtctgaactggag

SEQ ID NO: 18 - DNA, 34 bp, Artificial Sequence
  MRP 8 primer
gagagaagtactctactccttgtggctgtctttg